(19) United States
(12) Patent Application Publication
(10) Pub. No.: US 2024/0018221 A1
(43) Pub. Date: Jan. 18, 2024

(54) ANTIGEN BINDING MOLECULES AND USES THEREOF

(71) Applicant: Agency for Science, Technology and Research, Singapore (SG)

(72) Inventors: Wey Jia FONG, Singapore (SG); Boon Hwa CHOO, Singapore (SG); Mei Yee Vanessa DING, Singapore (SG); Chui Ping CHIN, Singapore (SG)

(73) Assignee: Agency for Science, Technology and Research, Singapore (SG)

(21) Appl. No.: 18/034,289

(22) PCT Filed: Oct. 26, 2021
(86) PCT No.: PCT/SG2021/050653

§ 371 Date: Apr. 27, 2023

(30) Foreign Application Priority Data
Oct. 27, 2020 (SG) 10202010654X

(51) Int. Cl. C07K 16/18; A61P 35/00

(52) U.S. Cl. CPC C07K 16/18; A61K 2039/505; A61P 35/00

(57) ABSTRACT

The invention relates generally to antigen-binding molecules that specifically bind to Myosin Heavy Chain 9 (MYH9) and uses thereof for the treatment of specific cancers, such as mast cell tumors. In one embodiment, the MYH9 is canine MYH9 and the antigen-binding molecule is an antibody. In another embodiment, the antigen-binding molecule is conjugated to a toxin, for the treatment of canine mast cell tumors.

### FIELD OF INVENTION

The invention relates generally to antigen-binding molecules. In particular, the present disclosure relates to antigen-binding molecules that specifically bind to Myosin Heavy Chain 9 (MYH9) and uses thereof for the treatment of specific cancers, such as mast cell tumors.

### BACKGROUND

Cancer in companion animals is a key concern for both veterinarians and pet owners as it is a major diagnosis as well as leading cause of death in 47% of the dogs and 32% of the cats. Some common types of cancer in companion animals include skin, lymphoma, hemangiosarcoma and mammary cancer. Despite the willingness of pet owners to pay and pursue treatments, cancer therapies for companion animals remain challenging due to lack of targeted oncology drugs for animal use.

Canine mast cell tumors (cMCTs), for example, are the most frequently diagnosed malignant skin neoplasms in dogs, representing up to 20% of canine cutaneous neoplasms. They are most commonly observed in dermal and subcutaneous tissues on the trunk and limbs. The clinical and histological staging of cMCTs are important prognostic factors for predicting the aggressiveness of the disease as well as survival of the patients. Treatment of cMCTs is also dependent on the staging of the disease. Surgical excision is the treatment of choice for dogs with low grade cMCTs. However, dogs with high grade aggressive cMCTs or evidence of spread may require systemic therapy after surgical excision. Several drugs that are currently available to treat these patients include high dose steroids, traditional chemotherapy (vinblastine, lomustine) and tyrosine kinase inhibitors (Palladia). These therapeutic approaches confer moderate efficacy (38-60%) while rendering patients to several harmful side effects such as gastrointestinal tract ulcerations and bone marrow suppression. Hence, there is a demand for a safer and more efficacious treatment for cMCTs.

Accordingly, it is generally desirable to overcome or ameliorate one or more of the above mentioned difficulties.

### SUMMARY

Disclosed herein is an antigen-binding molecule that specifically binds to Myosin Heavy Chain 9 (MYH9).

Disclosed herein is a chimeric molecule comprising an antigen-binding molecule as defined herein and a heterologous moiety.

Disclosed herein is an isolated polynucleotide comprising a nucleic acid sequence encoding the antigen-binding molecule as defined herein, or the chimeric molecule as defined herein.

Disclosed herein is a construct comprising a polynucleotide as defined herein in operable connection with one or more control sequences.

Disclosed herein is a host cell that contains the construct as defined herein.

Disclosed herein is a pharmaceutical composition comprising an antigen-binding molecule as defined herein or a chimeric molecule as defined herein.

Disclosed herein is an antigen-binding molecule as defined herein, a chimeric molecule as defined herein or a pharmaceutical composition as defined herein for use as a medicament.

Disclosed herein is a method for inhibiting proliferation and/or viability of a cancer cell, the method comprising contacting the cancer cell with a therapeutically effective amount of an antigen-binding molecule as defined herein, a chimeric molecule as defined herein or a pharmaceutical composition as defined herein, wherein the cancer cell is a cancer cell selected from a mast cell tumor, a mammary carcinoma, a hepatocellular carcinoma, a urothelial carcinoma, a histiocytic sarcoma, a Leydig cell tumor or a seminoma.

Disclosed herein is a method of reducing or inhibiting proliferation, survival and/or viability of a cancer in a subject, the method comprising administering a therapeutically effective amount of an antigen-binding molecule as defined herein, a chimeric molecule as defined herein or a pharmaceutical composition as defined herein to the subject, wherein the cancer is a cancer selected from a mast cell tumor, a mammary carcinoma, a hepatocellular carcinoma, a urothelial carcinoma, a histiocytic sarcoma, a Leydig cell tumor or a seminoma.

Disclosed herein is a method of treating a cancer in a subject, the method comprising administering a therapeutically effective amount of an antigen-binding molecule as defined herein, a chimeric molecule as defined herein or a pharmaceutical composition as defined herein to the subject, wherein the cancer is a cancer elected from a mast cell tumor, a mammary carcinoma, a hepatocellular carcinoma, a urothelial carcinoma, a histiocytic sarcoma, a Leydig cell tumor or a seminoma.

Disclosed herein is a method of treating a disease or condition associated with an undesired expression of MYH9 in a subject, wherein the method comprises administering a therapeutically effective amount of an antigen-binding molecule as defined herein, a chimeric molecule defined herein or a pharmaceutical composition as defined herein to the subject.

Disclosed herein is a method of detecting the likelihood of the presence of a cancer in a subject, the method comprising determining the level of MYH9 in a sample obtained from the subject, wherein an increased level of MYH9 as compared to a reference indicates the likelihood of the presence of a cancer in the subject, wherein the cancer is a cancer selected from a mast cell tumor, a mammary carcinoma, a hepatocellular carcinoma, a urothelial carcinoma, a histiocytic sarcoma, a Leydig cell tumor or a seminoma.

Disclosed herein is a method for predicting the prognosis of a cancer in a subject, the method comprising determining the level of MYH9 in a sample obtained from the subject, wherein an increased level of MYH9 as compared to a reference indicates a likelihood of a poor prognosis associated with tumor invasiveness in the subject, wherein the cancer is a cancer selected from a mast cell tumor, a mammary carcinoma, a hepatocellular carcinoma, a urothelial carcinoma, a histiocytic sarcoma, a Leydig cell tumor or a seminoma.

### BRIEF DESCRIPTION OF DRAWINGS

Embodiments of the present invention are hereafter described, by way of non-limiting example only, with reference to the accompanying drawings in which:

FIG. 1. Reactivity profile of mIgG_12B6 with various types of canine cancer and normal cell lines. (A) mIgG_12B6 was isotyped as mouse IgG1 with kappa light chain using the IsoStrip Mouse Monoclonal Antibody Isotyping Kit. (Roche) (B) Reactivity of mIgG_12B6 to canine cancer and normal cell lines were assessed via flow cytometry and graded 0-4 based on percentage of binding cell population. 0=no binding, 1<20% binding, 2=20%-40% binding, 3=40%-60% binding, 4 >60% binding. mIgG_12B6 binds specifically to mast tumor cell lines while retaining negligible reactivity with other types of cancer and normal cell lines

FIG. 2. IHC (Immunohistochemistry) staining of mIgG_12B6 on canine mast tumor tissue. Sections of 4 independent canine mast tumor specimens were stained with either mIgG_12B6 (E and H—grade 3, F and G—grade 2) or 10% goat serum (A to D) as negative control Immunohistochemistry (IHC) staining of canine mast cell tumor tissues with mIgG_12B6 showed substantial binding towards the malignant mast phenotype.

FIG. 3. Immunohistochemistry (IHC) staining of mIgG_12B6 on normal canine tissues. Cryosection of various types of normal canine tissues were stained with 3 μg/ml of mIgG_12B6. Negligible or no staining was observed. cMCT01 cryosection was stained in parallel as a positive control.

FIG. 4. Identification of mIgG_12B6 antigen. (A) Size of mIgG_12B6 antigen was determined via Western blot using whole cell lysate of cMCT01 cell line. The antigen was observed as a single band at ˜140 kDa under both reducing and non-reducing conditions. (B) Immunoprecipitation (IP) of mIgG_12B6 antigen using Protein G Phynexus tips. mIgG_12B6 IP eluate was simultaneously resolved via SDS-PAGE and Western blot. The antigen band at ˜140 kDa was excised from the SDS-PAGE gel and processed for protein identification via mass spectrometry analysis. (C) mIgG_12B6 antigen was identified as myosin-9 (MYH9) with approximately 40% peptide coverage. Peptide sequences identified by mass spectrometry were underlined.

FIG. 5. Validation of MYH9 as antigen target of mIgG_12B6 (A) A similar antigen band at ˜140 kDa was observed when mIgG_12B6 IP eluate was immunoblotted with mIgG_12B6 and anti-MYH9 commercial antibody. (B) Transient knockdown of MYH9 protein in cMCT01 cell line using MYH9 siRNA. Equal amounts of cell lysate protein were loaded in each lane as indicated by the similar levels of actin. SC: scrambled sequence; KD: knockdown with MYH9 targeting sequence; SF: Nucleofector reagent control. mIgG_12B6 binding was significant reduced after knockdown of MYH9 in cMCT01 cells using siRNA, thus confirming the antigen target of mIgG_12B6.

FIG. 6. mIgG_12B6 targeted a glycoprotein epitope (A) PNGaseF treatment removed the N-linked glycans in cMCT01 lysate, resulting in a loss of binding with mIgG_12B6. (B) Pronase treatment digested proteins in cMCT01 lysate into amino acids. The pronase treatment abolished binding of mIgG_12B6 with the antigen, suggesting that protein backbone is essential for reactivity. Taken together, these results indicate that the binding epitope of mIgG_12B6 comprises of both protein and glycan structures.

FIG. 7. Targeted delivery of SAP (saporin) or MMAE (auristatin) by mIgG_12B6 in vitro. cMCT01 (A) Cell cultures were treated with a single dose of mIgG_12B6—toxin complex at 2μg/ml for 48 hours. The toxins used are SAP and MMAE. The viabilities of the treated cultures were assessed using CellTitre Glo assay. Cytotoxic effect was observed in cMCT01 culture treated with mIgG_12B6-MMAE and mIgG_12B6-SAP complex, suggesting effective delivery of toxin into the cells. AA88 (B), a non-binding cell line, was subjected to the same treatment with minimal loss in viability, hence demonstrating specificity of toxin delivery by mIgG_12B6 to only binding cell lines.

FIG. 8: In-vivo efficacy of mIgG_12B6-SAP treatment in cMCT01 xenograft mouse model. Nude mice were injected subcutaneoously with cMCT01 cells and randomly categoried into 4 treatment groups (n=4-5 per group) consisting of PBS, mIgG_12B6, mIgG_isotype-SAP and mIgG_12B6-SAP. Treatments were administrated by intraperitoneal injection weekly for a period of 3 weeks (indicated by arrows) at a dose of 1.875mg/kg. Tumor volumes were monitored weekly over a period of 37 days. The tumor growth of mIgG_12B6-SAP treated mice was significantly inhibited as compared to control groups.

FIG. 9: Heavy and light variable sequences of mIgG_12B6. Complementarity-Determining Regions (CDR) are underlined

FIG. 10: Characterization of chimeric canine antibodies cIgG_{A}_12B6 and cIgG_{B}_12B6. mIgG_12B6 variable sequences were cloned into pTarget2.2 Hum vector containing constant canine IgG_{A} or IgG_{B} framework sequences. The chimeric antibodies were expressed in CHO cells and purified using Protein G resins. (A) Purified cIgG_{A}_12B6 and cIgG_{B}_12B6 resolved on SDS-PAGE gel (A) under both non-reducing and reducing conditions. (B) cIgG_{A}_12B6, cIgG_{B}_12B6 and mIgG_12B6 were dotted on nitrocellulose membrane and immunoblotted with rabbit anti-dog IgG. Reactivity was detected for the chimeric antibodies with anti-dog IgG whereas no signal was detected for its mouse equivalent. (C) Binding of chimeric 12B6 antibodies on canine cancer cell lines were evaluated via flow cytometry and graded 0-4 based on percentage of binding cell population. The chimeric antibodies retained similar binding profile as mIgG_12B6. (D) cMCT01 cells, pre-incubated with increasing amounts of mIgG_12B6, were treated with chimeric antibodies labelled with AlexaFluor488 and analyzed by flow cytometry. A decrease in mean fluorescence intensity was observed for both cIgG_{A}_12B6 and cIgG_{B}_12B6 in the presence of mIgG_12B6 due to competitive binding for the same epitope.

FIG. 11: cIgG_{B}_12B6FcX, the MALA PG variant of cIgG_{B}_12B6, reduces Fc-mediated binding to canine PBMCs. (A) Flow cytometry analysis of IgG_{B}_12B6-AF488 and IgG_{B}_12B6FcX-AF488 on canine PBMCs. IgG_{B}_12B6FcX exhibits lower reactivity with canine PBMCs than IgG_{B}_12B6 (B) Myeloid and normal mast cell populations in canine PBMCs (CD18-647 positive) exhibits lower binding for cIgG_{B}_12B6FcX compared to cIgG_{B}_12B6. PBMCs: Peripheral blood mononuclear cells. AF488: AlexaFluor 488

FIG. 12. Targeted delivery of toxin by chimeric canine cIgG_{B}_12B6FcX in vitro. (A) Varying concentrations of biotinylated chimeric antibodies were pre-incubated with streptavidin-saporin at molar ratio of 1:2 before adding them to cMCT01 cell cultures. Viabilities of treated cultures were subsequently assessed using CTG assay after 72 hours and luminescence readings obtained were normalized to buffer control. Comparable cytotoxicity effects were observed in dose-dependent manner for cultures treated with both chimeric antibodies-saporin complex. (B) cIgG_{B}_12B6FcX-MMAE was synthesized by directly conjugating MMAE to cIgG_{B}_12B6FcX with DAR ratio of 4. To assess the cytotoxicity of the conjugate, it was added to cMCT01 at concentrations ranging from 10⁻⁴ nM to 10² nM. The IC₅₀ values for cMCT01 was calculated as 0.02733 nM.

FIG. 13: In-vivo efficacy of cIgG_{B}_12B6FcX-SAP treatment in cMCT01 xenograft mouse model. Nude mice were injected subcutaneously with cMCT01 cells and randomised into 6 treatment groups (n=4-5 per group) when the tumors reached 150 mm³. The treatment groups consisting of PBS, cIgG_{B}_12B6FcX, free MMAE and cIgG_{B}_12B6FcX-MMAE at 5 mg/kg, 10 mg/kg and 20 mg/kg. Treatments were administrated by tail vein intravenous injection at intervals of 4 days (indicated by arrows). Tumor volumes were monitored weekly over a period of 78 days. Complete remission was observed in mice treated with cIgG_{B}_12B6FcX-MMAE.

FIG. 14: Immunohistochemistry (IHC) staining of mIgG_12B6 on canine/feline epithelial tumor tissue array. Various types of canine/feline tumor tissues were stained mIgG_12B6 (A) or 10% goat serum as negative control (B). Positive mIgG_12B6 staining was observed for grade 3 feline and canine mammary carcinoma (k1-k4), canine hepatocellular carcinoma (m1-m2) and canine urothelial carcinoma (n3-n4). The layout of tissue types in the array is depicted in (C).

FIG. 15: Immunohistochemistry (IHC) staining of mIgG_12B6 on canine mesenchymal and reproductive tumors tissue array. Various types of canine tumor tissues were stained mIgG_12B6 (A) or 10% goat serum as negative control (B). Positive mIgG_12B6 staining was observed for canine histiocytic sarcoma (15-16), Leydig cell tumor (17-18) and canine seminoma (m5-m6). The layout of tissue types in the array is depicted in (C).

FIG. 16. Serum clinical chemistry (B) and hematology analysis (A) of mice treated with a single dose of cIgGB_12B6FcX-MMAE at 5 mg/kg, 10 mg/kg and 15 mg/kg. (C) Incidence of lesions observed in tissues of treated mice. Comparison between control and treated groups were performed using two-tailed student's t-test. Blood urea nitrogen (BUN); Aspartate Aminotransferase (AST); Alanine aminotransferase (ALT); Total Bilirubin (TBIL).

### DETAILED DESCRIPTION

Disclosed herein is an antigen-binding molecule that specifically binds to Myosin Heavy Chain 9 (MYH9).

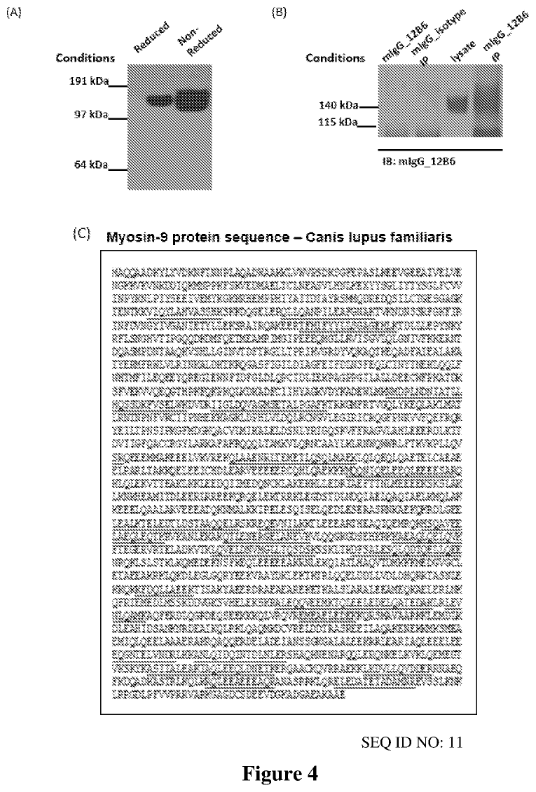

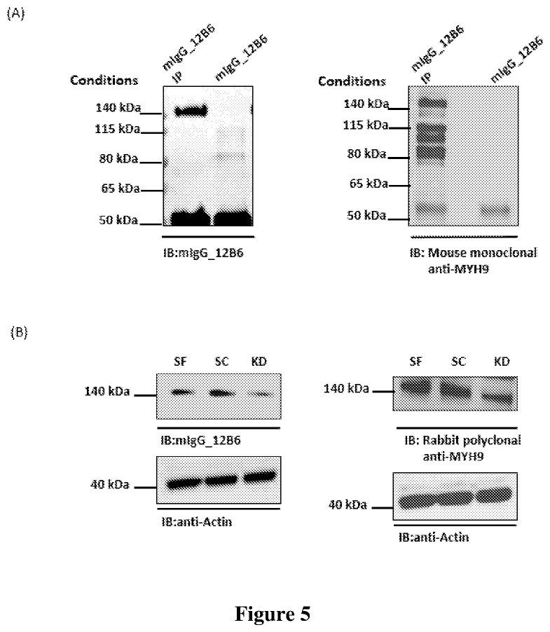

The antigen, MYH9, may also be referred to as Non-Muscle Myosin Heavy Chain Ha or Myosin Heavy Chain, type A. In one embodiment, the MYH9 is canine MYH9. In one embodiment, the MYH9 is a glycosylated MYH9. The antigen may be a peptide derived from MYH9. The amino acid sequence of MYH9 may, for example, be a sequence as shown in FIG. 4(c). In one embodiment, the peptide derived from MYH9 comprises an N-linked glycan.

Without being bound by theory, mIgG_12B6, targeting canine MYH-9 on canine cells, is highly specific to mast tumor cell lines and tissues. It demonstrates negligible to no staining on normal canine tissue and cell lines, making it safe for use as a therapeutic. As an antibody drug conjugate, mIgG_12B6-MMAE following internalization is able to deliver the toxin into reactive cells and induce killing Together with its high selectivity for mast cancer phenotype, mIgG_12B6 is a novel and potential candidate for mast cancer therapy in canine.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity. Representative antigen-binding molecules that are useful in the practice of the present invention include antibodies and their antigen-binding fragments. The term “antigen-binding molecule” includes antibodies and antigen-binding fragments of antibodies.

In an embodiment, the antigen-binding molecule, as described herein, is conjugated to another molecule or moiety, including functional moieties (e.g., toxins), detectable moieties (e.g., fluorescent molecules, radioisotopes), small molecule drugs and polypeptides.

The term “antibody”, as used herein, is understood to mean any antigen-binding molecule or molecular complex comprising at least one complementarity determining region (CDR) that binds specifically to, or interacts specifically with, the target antigen. The term “antibody” includes full-length immunoglobulin molecules comprising two heavy (H) chains and two light (L) chains inter-connected by disulfide bonds, as well as multimers thereof (e.g., IgM). Each heavy chain comprises a heavy chain variable region (which may be abbreviated as HCVR, VH or V_{H}) and a heavy chain constant region. The heavy chain constant region typically comprises three domains—C_{H}1, C_{H}2 and C_{H}3. Each light chain comprises a light chain variable region (which may be abbreviated as LCVR, VL, VK, V_{K} or V_{L}) and a light chain constant region. The light chain constant region will typically comprise one domain (C_{L}1). The V_{H} and V_{L} regions can be further subdivided into regions of hypervariability, termed complementarity determining regions (CDRs), interspersed with regions that are more conserved, also referred to as framework regions (FR). Each V_{H} and V_{L} typically comprises three CDRs and four FRs, arranged from amino-terminus to carboxy-terminus in the following order: FR1, CDR1, FR2, CDR2, FR3, CDR3, FR4. In some embodiments, the FRs of the antigen-binding molecules described herein may be identical to the FR of germline sequences of the target species (i.e., the species to which the antigen-binding molecules or antigen-binding fragments thereof, as described herein, will be administered). In some embodiments, the FR may be naturally or artificially modified. Whilst it is generally desirable that each of the FR sequences are identical to FR sequences derived from immunoglobulin molecules of the target species, including to minimize an immune response being raised against the binding molecule upon administration to a subject of the target species, in some embodiments, the antigen-binding molecule, or antigen-binding fragment thereof, may comprise one or more amino acid residues across one or more of its FR sequences that would be foreign at a corresponding position in one or more FR from the target species.

An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called α, δ, ϵ, γ, and μ, respectively. The subunit structures and three-dimensional configurations of different classes of immunoglobulins are well known to a person skilled in the art.

As used herein, the term “complementarity determining regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined for example by Kabat (i.e., about residues 24-34 (L1), 50-56 (L2) and 89-97 (L3) in the light chain variable domain and 31-35 (H1), 50-65 (H2) and (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop.

An “antigen-binding site” refers to the site, i.e., one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site. An antigen-binding site of an antigen-binding molecule described herein typically binds specifically to an antigen and more particularly to an epitope of the antigen.

The present disclosure extends to antigen-binding molecules that bind specifically to MYH9 of any species. The MYH9 may be canine MYH9 or feline MYH9. In an embodiment, the MYH9 is canine MYH9. In another embodiment, the MYH9 is feline MYH9. The present disclosure extends to antigen binding molecules that bind specifically to native MYH9 (i.e., naturally-occurring MYH9), as well as to variants thereof. Such variants may include MYH9 molecules that differ from a naturally-occurring (wild-type) molecule by one or more amino acid substitutions, deletions and/or insertions. Variant MYH9 molecules of this type may be naturally-occurring or synthetic (e.g., recombinant) forms. It is to be understood, however, that in one embodiment, the antigen-binding molecules described herein bind specifically to a native form of MYH9.

The terms “antigen-binding fragment”, “antigen-binding portion”, “antigen-binding domain” and “antigen-binding site” are used interchangeably herein to refer to a part of an antigen-binding molecule that participates in antigen-binding. These terms include any naturally occurring, enzymatically obtainable, synthetic, or genetically engineered polypeptide or glycoprotein that specifically binds an antigen to form a complex.

Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, one-armed antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}—V_{H}, V_{H}—V_{L} or V_{L}—V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}—C_{H}1; (ii) V_{H}—C_{H}2; (iii) V_{H}—C_{H}3; (iv) V_{H}—C_{H}1—C_{H}2; (v) V_{H}—C_{H}1—C_{H}2—C_{H}3, (vi) V_{H}—C_{H}2—C_{H}3; (vii) V_{H}—C_{L}; (viii) V_{L}—C_{H}1; (ix) V_{L}—C_{H}2, (x) V_{L}—C_{H}3; (xi) V_{L}—C_{H}1—C_{H}2; (xii) V_{L}—C_{H}1—C_{H}2—C_{H}3; (xiii) V_{L}—C_{H}2—C_{H}3; and (xiv) V_{L}—C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V H or V L domain (e.g., by disulfide bond(s)). A multispecific antigen-binding molecule will typically comprise at least two different variable domains, wherein each variable domain is capable of specifically binding to a separate antigen or to a different epitope on the same antigen. Any multispecific antigen-binding molecule format, including bispecific antigen-binding molecule formats, may be adapted for use in the context of an antigen-binding fragment of an antibody of the present disclosure using routine techniques available in the art.

The term “variable region” or “variable domain” refers to the domain of an antibody heavy or light chain that is involved in binding the antigen binding molecule to antigen. The variable domains of the heavy chain and light chain (V_{H} and V_{L}, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single V_{H} or V_{L} domain may be sufficient to confer antigen-binding specificity.

The term “constant domains” or “constant region” as used herein denotes the sum of the domains of an antibody other than the variable region. The constant region is not directly involved in binding of an antigen, but exhibits various immune effector functions.

In one embodiment, the antigen-binding molecule or antigen-binding fragment thereof is modified for compatibility with the target species. Thus, in an embodiment, the antigen-binding molecule or antigen-binding fragment thereof is caninized or felinized.

By “caninized” is meant that the antigen-binding molecule comprises an amino acid sequence that is compatible with canine, such that the amino acid sequence is unlikely to be seen as foreign by the immune system of a canine subject. In an embodiment, the caninized antigen-binding molecule comprises one or more immunoglobulin framework regions derived from one or more canine immunoglobulin molecules. In some embodiments, all of the framework regions of the caninized antigen-binding molecule will be derived from one or more canine immunoglobulin molecules. The caninized antibody may optionally comprise an immunoglobulin heavy chain constant region derived from a canine immunoglobulin molecule.

By “felinized” is meant that the antigen-binding molecule comprises an amino acid sequence that is compatible with feline, such that the amino acid sequence is unlikely to be seen as foreign by the immune system of a feline subject. In an embodiment, the felinized antigen-binding molecule comprises one or more immunoglobulin framework regions derived from one or more feline immunoglobulin molecules. In some embodiments, all of the framework regions of the felinized antigen-binding molecule will be derived from one or more feline immunoglobulin molecules. The felinized antibody may optionally comprise an immunoglobulin heavy chain constant region derived from a feline immunoglobulin molecule.

It is to be understood that the present disclosure also extends to antigen-binding molecules that are compatible with species other than canine and feline species. In this context, the antigen-binding molecules can be referred to as “speciesized”, referring to the target species to which the molecule will be administered.

Suitable methods of designing and producing recombinant antibodies or antigen-binding molecules that are compatible with the target species will be familiar to persons skilled in the art, illustrative examples of which are described in Cattaneo (2010; supra), WO 2006/131951, WO 2012/153122, WO 2013/034900, WO 2012/153121 and WO 2012/153123, the contents of which are incorporated herein by reference in their entirety.

The phrase “specifically binds” or “specific binding” refers to a binding reaction between two molecules that is at least two times the background and more typically more than 10 to 100 times background molecular associations under physiological conditions. When using one or more detectable binding agents that are proteins, specific binding is determinative of the presence of the protein, in a heterogeneous population of proteins and other biologics. Thus, under designated immunoassay conditions, the specified antigen-binding molecule binds to a particular antigenic determinant, thereby identifying its presence. Specific binding to an antigenic determinant under such conditions requires an antigen-binding molecule that is selected for its specificity to that determinant. This selection may be achieved by subtracting out antigen-binding molecules that cross-react with other molecules. A variety of immunoassay formats may be used to select antigen-binding molecules (e g., immunoglobulins) such that they are specifically immunoreactive with a particular antigen. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Methods of determining binding affinity and specificity are also well known in the art (see, for example, Harlow and Lane, supra); Friefelder, “Physical Biochemistry: Applications to biochemistry and molecular biology” (W.H. Freeman and Co. 1976)).

“Affinity” or “binding affinity” refers to the strength of the sum total of non-covalent interactions between a single binding site of a molecule (e.g., an antigen-binding molecule) and its binding partner (e.g., an antigen). Unless indicated otherwise, as used herein, “binding affinity” refers to intrinsic binding affinity which reflects a 1:1 interaction between members of a binding pair e.g., an antigen-binding molecule. The affinity of a molecule X for its partner Y can generally be represented by the dissociation constant (Kd), which is the ratio of dissociation and association rate constants (k_{off} and kₒₙ, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

The terms “polypeptide”, “peptide”, or “protein” are used interchangeably herein to designate a linear series of amino acid residues connected one to the other by peptide bonds between the alpha-amino and carboxy groups of adjacent residues. The amino acid residues are usually in the natural “L” isomeric form. However, residues in the “D” isomeric form can be substituted for any L-amino acid residue, as long as the desired functional property is retained by the polypeptide

As used herein, the term “modified antibody” includes synthetic forms of antibodies which are altered such that they are not naturally occurring, e.g., antibodies that comprise at least two heavy chain portions but not two complete heavy chains (such as domain deleted antibodies or minibodies); multispecific forms of antibodies (e.g., bispecific, trispecific, etc.) altered to bind to two or more different antigens or to different epitopes on a single antigen; heavy chain molecules joined to scFv molecules and the like. ScFv molecules are known in the art and are described, e.g., in U.S. Pat. No. 5,892,019. In addition, the term “modified antibody” includes multivalent forms of antibodies (e.g., trivalent, tetravalent, etc., antibodies that bind to three or more copies of the same antigen).

The antigen-binding molecule may comprise a heavy chain variable (VH) region comprising the VHCDR1 amino acid sequence GYSITSDYAWN (SEQ ID NO: 1), the VHCDR2 amino acid sequence YISYSGSTNYNPSLKS (SEQ ID NO: 2) and the VHCDR3 amino acid sequence NPPFVY (SEQ ID NO: 3). The antigen-binding molecule may comprise a light chain variable (VL) region comprising the VLCDR1 amino acid sequence TASSGVSSGYLH (SEQ ID: 4), the VLCDR2 amino acid sequence STSNLAS (SEQ ID NO: 5) and the VLDR3 amino acid sequence HQYHRSPFT (SEQ ID NO: 6).

In one embodiment, the antigen-binding molecule comprises:
a) a heavy chain variable (VH) region comprising the VHCDR1 amino acid sequence GYSITSDYAWN (SEQ ID NO: 1), the VHCDR2 amino acid sequence YISYSGSTNYNPSLKS (SEQ ID NO: 2) and the VHCDR3 amino acid sequence NPPFVY (SEQ ID NO: 3); and
b) a light chain variable (VL) region comprising the VLCDR1 amino acid sequence TASSGVSSGYLH (SEQ ID: 4), the VLCDR2 amino acid sequence STSNLAS (SEQ ID NO: 5) and the VLDR3 amino acid sequence HQYHRSPFT (SEQ ID NO: 6).

The antigen-binding molecule may comprise a heavy chain variable region comprising an amino acid sequence having at least 70% (or at least 75%, 80%, 85%, 90% or 95%) sequence identity to:

(SEQ ID NO: 7)
QVQLQESGPGLVKPSQSLSLTCTVTGYSITSDYAWNWLRQFPGNKLEWM
GYISYSGSTNYNPSLKSRISITRDTSKNQFFLQLNSVTTEDTATYYCAR
NPPFVYWGQGTLVTVST.

The antigen-binding molecule may comprise a light chain variable region comprising an amino acid sequence having at least 70% (or at least 75%, 80%, 85%, 90% or 95%) sequence identity to:

(SEQ ID NO: 8)
QIVLTQSPAIMSASLGERVTMTCTASSGVSSGYLHWYQQKPGSSPKLWI
YSTSNLASGVPARFSGSGSGTSYSLTISSMEAEDAATYYCHQYHRSPFT
FGSGTKLEIERADAAPTVS.

In one embodiment, the antigen-binding molecule comprises:
a) a heavy chain variable region comprising an amino acid sequence having at least 70% (or at least 75%, 80%, 85%, 90% or 95%) sequence identity to QVQLQESGPGLVKPSQSLSLTCTVTGYSITSDYAWNWLRQFPGNKLEWMGYISY SGSTNYNPSLKSRISITRDTSKNQFFLQLNSVTTEDTATYYCARNPPFVYWGQGT LVTVST (SEQ ID NO: 7); and
b) a light chain variable region comprising an amino acid sequence having at least 70% (or at least 75%, 80%, 85%, 90% or 95%) sequence identity to

(SEQ ID NO: 8)
QIVLTQSPAIMSASLGERVTMTCTASSGVSSGYLHWYQQKPGSSPKLWI
YSTSNLASGVPARFSGSGSGTSYSLTISSMEAEDAATYYCHQYHRSPFT
FGSGTKLEIERADAAPTVS.

In one embodiment, the antigen-binding molecule comprises
a) a heavy chain variable region (VH) as defined herein comprising at least 70% sequence identity to at least one region other than a CDR of the VH amino acid sequence set forth in SEQ ID NO:7 (e.g., to at least one framework region, such as 1, 2, 3 or 4 framework regions, of the VH), and
b) a light chain variable region (VL) as defined herein comprising at least 70% sequence identity to at least one region other than a CDR of the VL amino acid sequence set forth in SEQ ID NO:8 (e.g., to at least one framework region, such as 1, 2, 3 or 4 framework regions, of the VL).

In one embodiment, the antigen-binding molecule comprises
a) a VH as defined herein which is distinguished from the VH amino acid sequence set forth in SEQ ID NO:7 by a deletion, substitution or addition of one or more (e.g., 1, 2, 3, 4 or 5) amino acids in at least one region other than a CDR of the VH amino acid sequence set forth in SEQ ID NO:7 (e.g., in at least one framework region, such as in 1, 2, 3 or 4 framework regions, of the VH), and
b) a VL as defined in (1) which is distinguished from the VL amino acid sequence set forth in SEQ ID NO:8 by a deletion, substitution or addition of one or more (e.g., 1, 2, 3, 4 or 5) amino acids in at least one region other than a CDR of the VL amino acid sequence set forth in SEQ ID NO:8 (e.g., in at least one framework region, such as in 1, 2, 3 or 4 framework regions, of the VL).

In one embodiment, the antigen-binding molecule comprises:
a) a heavy chain comprising an amino acid sequence having at least 70% (or at least 75%, 80%, 85%, 90% or 95%) sequence identity to:

(SEQ ID NO: 9)
QVQLQESGPGLVKPSQSLSLTCTVTGYSITSDYAWNWLRQFPGNKLEWM
GYISYSGSTNYNPSLKSRISITRDTSKNQFFLQLNSVTTEDTATYYCAR
NPPFVYWGQGTLVTVSTASTTAPSVFPLAPSCGSTSGSTVALACLVSGY
FPEPVTVSWNSGSLTSGVHTFPSVLQSSGLYSLSSMVTVPSSRWPSETF
TCNVAHPASKTKVDKPVPKRENGRVPRPPDCPKCPAPEMLGGPSVFIFP
PKPKDTLLIARTPEVTCVVVDLDPEDPEVQISWFVDGKQMQTAKTQPRE
EQFNGTYRVVSVLPIGHQDWLKGKQFTCKVNNKALPSPIERTISKARGQ
AHQPSVYVLPPSREELSKNTVSLTCLIKDFFPPDIDVEWQSNGQQEPES
KYRTTPPQLDEDGSYFLYSKLSVDKSRWQRGDTFICAVMHEALHNHYTQ
ESLSHSPGK;

and/or
b) a light chain comprising an amino acid sequence having at least 70% (or at least 75%, 80%, 85%, 90% or 95%) sequence identity to:

(SEQ ID NO: 10)
QIVLTQSPAIMSASLGERVTMTCTASSGVSSGYLHWYQQKPGSSPKLWI
YSTSNLASGVPARFSGSGSGTSYSLTISSMEAEDAATYYCHQYHRSPFT
FGSGTKLEIERTDAQPAVYLFQPSPDQLHTGSASVVCLLNSFYPKDINV
KWKVDGVIQDTGIQESVTEQDKDSTYSLSSTLTMSSTEYLSHELYSCEI
THKSLPSTLIKSFQRSECQRVD.

In one embodiment, the antigen-binding molecule comprises:
a) a heavy chain comprising an amino acid sequence having at least 70% (or at least 75%, 80%, 85%, 90% or 95%) sequence identity to:

(SEQ ID NO: 11)
QVQLQESGPGLVKPSQSLSLTCTVTGYSITSDYAWNWLRQFPGNKLEWM
GYISYSGSTNYNPSLKSRISITRDTSKNQFFLQLNSVTTEDTATYYCAR
NPPFVYWGQGTLVTVSTASTTAPSVFPLAPSCGSTSGSTVALACLVSGY
FPEPVTVSWNSGSLTSGVHTFPSVLQSSGLYSLSSMVTVPSSRWPSETF
TCNVAHPASKTKVDKPVPKRENGRVPRPPDCPKCPAPEAAGGPSVFIFP
PKPKDTLLIARTPEVTCVVVDLDPEDPEVQISWFVDGKQMQTAKTQPRE
EQFNGTYRVVSVLPIGHQDWLKGKQFTCKVNNKALGSPIERTISKARGQ
AHQPSVYVLPPSREELSKNTVSLTCLIKDFFPPDIDVEWQSNGQQEPES
KYRTTPPQLDEDGSYFLYSKLSVDKSRWQRGDTFICAVMHEALHNHYTQ
ESLSHSPGK;

and/or
b) a light chain comprising an amino acid sequence having at least 70% (or at least 75%, 80%, 85%, 90% or 95%) sequence identity to:

(SEQ ID NO: 10)
QIVLTQSPAIMSASLGERVTMTCTASSGVSSGYLHWYQQKPGSSPKLWI
YSTSNLASGVPARFSGSGSGTSYSLTISSMEAEDAATYYCHQYHRSPFT
FGSGTKLEIERTDAQPAVYLFQPSPDQLHTGSASVVCLLNSFYPKDINV
KWKVDGVIQDTGIQESVTEQDKDSTYSLSSTLTMSSTEYLSHELYSCEI
THKSLPSTLIKSFQRSECQRVD.

The antigen-binding molecule as defined herein may comprise one or more conservative amino acid substitutions.

A “conservative amino acid substitution” is to be understood as meaning a substitution in which the amino acid residue is replaced with an amino acid residue having a similar side chain Families of amino acid residues having similar side chains have been defined in the art, which can be generally sub-classified as shown in the table “Amino Acid Classification”, below:

### Amino Acid Sub-Classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, Histidine |
| Small | Glycine, Serine, Alanine, Threonine, Proline |
| Polar/neutral | Asparagine, Histidine, Glutamine, Cysteine, Serine, |
| | Threonine |
| Polar/large | Asparagine, Glutamine |
| Hydrophobic | Tyrosine, Valine, Isoleucine, Leucine, Methionine, |
| | Phenylalanine, Tryptophan |
| Aromatic | Tryptophan, Tyrosine, Phenylalanine |
| Residues that | Glycine and Proline |
| influence chain | |
| orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional polypeptide can readily be determined by assaying its activity.

Conservative substitutions are also shown in the table below (EXEMPLARY AND PREFERRED AMINO ACID SUBSTITUTIONS) Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants can be screened for their ability to bind specifically to NGF using methods known to persons skilled in the art, including those methods described elsewhere herein.

### Exemplary and Preferred Amino Acid Substitutions

| | | Preferred |
|---|---|---|
| Original Residue | Exemplary Substitutions | Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

The antigen-binding molecule may be a monoclonal, recombinant or polyclonal antibody. It may be chimeric or caninized The antigen-binding molecule may include a bispecific and hetero-conjugated antibody. It may be a chimeric antigen receptor (CAR). It may be a single variable domain, a domain antibody, an antigen binding fragment, an immunologically effective fragment, a single chain Fv, a single chain antibody, a univalent antibody lacking a hinge region, a minibody, or a diabody.

In one embodiment, the antigen-binding molecule is an antibody or antigen-binding fragment thereof.

In one embodiment, the antibody or antigen-binding molecule thereof is caninized or chimerized.

The antibody or antigen binding fragment thereof may be a full-length antibody, a substantially intact antibody, a Fab fragment, a scFab, a Fab′, a single chain variable fragment (scFv) or a one-armed antibody.

Disclosed herein is a chimeric molecule comprising an antigen-binding molecule as defined herein and a heterologous moiety.

As used herein, a “chimeric” molecule is one which comprises one or more unrelated types of components or contain two or more chemically distinct regions which can be conjugated to each other, fused, linked, translated, attached via a linker, chemically synthesized, expressed from a nucleic acid sequence, etc. For example, a peptide and a nucleic acid sequence, a peptide and a detectable label, unrelated peptide sequences, and the like. In embodiments in which the chimeric molecule comprises amino acid sequences of different origin, the chimeric molecule includes (1) polypeptide sequences that are not found together in nature (i.e., at least one of the amino acid sequences is heterologous with respect to at least one of its other amino acid sequences), or (2) amino acid sequences that are not naturally adjoined. For example, a “chimeric” antibody” as used herein refers to an antibody in which a portion of the heavy and/or light chain is derived from a particular source or species, while the remainder of the heavy and/or light chain is derived from a different source or species.

The heterologous moiety may be a detectable moiety, a half-life extending moiety or a therapeutic moiety.

Detectable moieties contemplated by the present invention include for example any species known in the art that is appropriate for diagnostic detection, including in vitro detection and in vivo imaging. The detectable moiety may be, for example, a fluorophore, a radionuclide reporter, a metal-containing nanoparticle or microparticle, an ultrasound contrast agent (e.g., a nanobubble or microbubble) or an optical imaging dye. This also includes contrast particles visible in magnetic resonance imaging (MRI) and magnetic particle imaging (MPI). Fluorophores can be detected and/or imaged, for example, by fluorescence polarization, fluorescence-activated cell sorting and fluorescence microscopy, which may or may not be in combination with electrospray ionization-mass spectrometry (ESI-MS) detection, as well as fluorescence emission computed tomography (FLECT) imaging. Radionuclide reporters can be detected and imaged by radionuclide (nuclear) detection, such as, for example, single-photon emission computed tomography (SPECT), positron emission tomography (PET) or scintigraphic imaging. Metal-containing nanoparticles or microparticles may be detected using optical imaging, including MRI, which is typically used with paramagnetic nanoparticles or microparticles, and MPI, which is generally used with superparamagnetic particles. Ultrasound contrast agents can be detected using ultrasound imaging including contrast-enhanced ultrasound (CEU).

The detectable label may also be an enzyme-substrate label. The enzyme may generally catalyze a chemical alteration of the chromogenic substrate that can be measured using various techniques. For example, the enzyme may catalyze a chemical alteration of the chromogenic substrate that can be measured using the various techniques. For example, the example may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as unease and xanthine oxidase), lactoperoxidase, microperoxidase, and the like.

Examples of enzyme-substrate combinations include, for example:
1) Horseradish peroxidase (HRPO) utilizes hydrogen peroxide to oxidize a dye precursor (e.g., orthophenylene diamine (OPD) or 3,3′,5,5′-tetramethyl benzidine hydrochloride (TMB));
2) alkaline phosphatase (AP) with para-Nitrophenyl phosphate as chromogenic substrate; and
β-D-galactosidase (β-D-Gal) with a chromogenic substrate (e.g., p-nitrophenyl-β-D-galactosidase) or fluorogenic substrate 4-methylumbelliferyl-β-D-galactosidase.

In another embodiment of the invention, the antigen-binding molecule need not be labeled, and the presence thereof can be detected using a labeled antibody which binds to the antigen-binding molecule. The antigen-binding molecule of the present invention may be employed in any known assay method, such as competitive binding assays, direct and indirect sandwich assays, immunohistochemistry and immunoprecipitation assays.

In one embodiment, the chimeric molecule comprises at least one heterologous moiety that is a “half-life extending moiety”. Half-life extending moieties, can comprise, for example, (i) XTEN polypeptides; (ii) Fc; (iii) albumin, (iv) albumin binding polypeptide or fatty acid, (v) the C-terminal peptide (CTP) of the 13 subunit of human chorionic gonadotropin, (vi) PAS; (vii) HAP; (viii) transferrin; (ix) polyethylene glycol (PEG); (x) hydroxyethyl starch (HES), (xi) polysialic acids (PSAs); (xii) a clearance receptor or fragment thereof which blocks binding of the chimeric molecule to a clearance receptor; (xiii) low complexity peptides; (xiv) or any combinations thereof. In some embodiments, the half-life extending moiety comprises an Fc region. In other embodiments, the half-life extending moiety comprises two Fc regions fused by a linker. Exemplary heterologous moieties also include, e.g., FcRn binding moieties (e.g., complete Fc regions or portions thereof which bind to FcRn), single chain Fc regions (scFc regions, e.g., as described in U.S. Publ. No. 20080260738, WO 2008/012543 and WO 2008/1439545), or processable scFc regions. In some embodiments, a heterologous moiety can include an attachment site for a non-polypeptide moiety such as polyethylene glycol (PEG), hydroxyethyl starch (HES),

In one embodiment, the therapeutic moiety is a toxin. The toxin may, for example, be monomethyl auristatin E (MMAE), mertansine (DM-1), saporin, gemcitabine, irinotecan, etoposide, vinblastine, pemetrexed, docetaxel, paclitaxel, platinum agents (for example, cisplatin, oxaliplatin or carboplatin), vinorelbine, capecitabine, mitoxantrone, ixabepilone, eribulin, 5-fluorouracil, trifluridine or tipiracil.)

In one embodiment, the toxin is auristatin (e.g. monomethyl auristatin E (MMAE)) or saporin.

In one embodiment, the antigen-binding molecule is joined to the therapeutic moiety via a valine-citruline p-aminobenzyloxycarbonyl (VC-PAB linker). In one embodiment, the linker is cleavable. In one embodiment, the linker is cathepsin B cleavable.

In one embodiment, the chimeric molecule is an Antibody Drug Conjugate (ADC).

In one embodiment, the antigen-binding molecule or chimeric molecule as defined herein is capable of being internalized into a cell. This makes the antigen-binding molecule or chimeric molecule suitable for delivering a therapeutic moiety into a cell.

Disclosed herein is an isolated polynucleotide comprising a nucleic acid sequence encoding the antigen-binding molecule as defined herein, or the chimeric molecule as defined herein.

The term “polynucleotide” or “nucleic acid” are used interchangeably herein to refer to a polymer of nucleotides, which can be mRNA, RNA, cRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA.

Also disclosed herein is a vector that comprises a nucleic acid encoding the antigen-binding molecule as described herein.

By “vector” is meant a nucleic acid molecule, preferably a DNA molecule derived, for example, from a plasmid, bacteriophage, or virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a mini-chromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

Disclosed herein is a construct comprising a polynucleotide as defined herein in operable connection with one or more control sequences.

The term “construct” refers to a recombinant genetic molecule including one or more isolated nucleic acid sequences from different sources. Thus, constructs are chimeric molecules in which two or more nucleic acid sequences of different origin are assembled into a single nucleic acid molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Constructs of the present invention will generally include the necessary elements to direct expression of a nucleic acid sequence of interest that is also contained in the construct, such as, for example, a target nucleic acid sequence or a modulator nucleic acid sequence. Such elements may include control elements or regulatory sequences such as a promoter that is operably linked to (so as to direct transcription of) the nucleic acid sequence of interest, and often includes a polyadenylation sequence as well. Within certain embodiments of the invention, the construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a host cell. Two or more constructs can be contained within a single nucleic acid molecule, such as a single vector, or can be containing within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in an organism or part thereof including a host cell. For the practice of the present invention, conventional compositions and methods for preparing and using constructs and host cells are well known to one skilled in the art, see for example, Molecular Cloning: A Laboratory Manual, 3rd edition Volumes 1, 2, and 3. J. F. Sambrook, D. W. Russell, and N. Irwin, Cold Spring Harbor Laboratory Press, 2000.

By “control element”, “control sequence”, “regulatory sequence” and the like, as used herein, mean a nucleic acid sequence (e.g., DNA) necessary for expression of an operably linked coding sequence in a particular host cell. The control sequences that are suitable for prokaryotic cells for example, include a promoter, and optionally a cis-acting sequence such as an operator sequence and a ribosome binding site. Control sequences that are suitable for eukaryotic cells include transcriptional control sequences such as promoters, polyadenylation signals, transcriptional enhancers, translational control sequences such as translational enhancers and internal ribosome binding sites (IRES), nucleic acid sequences that modulate mRNA stability, as well as targeting sequences that target a product encoded by a transcribed polynucleotide to an intracellular compartment within a cell or to the extracellular environment.

Disclosed herein is a host cell that contains the construct as defined herein.

The terms “host”, “host cell”, “host cell line” and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the antigen binding molecules of the present invention. Host cells include cultured cells, e.g., mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

Disclosed herein is a pharmaceutical composition comprising an antigen-binding molecule as defined herein or a chimeric molecule as defined herein.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Representative pharmaceutically acceptable carriers include any and all solvents, dispersion media, coatings, surfactants, antioxidants, preservatives {e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient(s), its use in the pharmaceutical compositions is contemplated.

The pharmaceutical compositions may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Suitable pharmaceutical compositions may be administered intravenously, subcutaneously or intramuscularly. In some embodiments, the compositions are in the form of injectable or infusible solutions. A preferred mode of administration is parenteral (e.g., intravenous, subcutaneous, intraperitoneal, intramuscular). In specific embodiments, the pharmaceutical composition is administered by intravenous infusion or injection. In other embodiments, the pharmaceutical composition is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like.

Preservatives and other additives can also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin and/or by the maintenance of the required particle size. In specific embodiments, an agent of the present disclosure may be conjugated to a vehicle for cellular delivery. In these embodiments, the agent may be encapsulated in a suitable vehicle to either aid in the delivery of the agent to target cells, to increase the stability of the agent, or to minimize potential toxicity of the agent. As will be appreciated by a skilled artisan, a variety of vehicles are suitable for delivering an agent of the present disclosure. Non-limiting examples of suitable structured fluid delivery systems may include nanoparticles, liposomes, microemulsions, micelles, dendrimers and other phospholipid-containing systems. Methods of incorporating agents of the present disclosure into delivery vehicles are known in the art. Although various embodiments are presented below, it will be appreciated that other methods known in the art to incorporate an antigen-binding molecule, as described herein, into a delivery vehicle are contemplated.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. An antigen-binding molecule of the present disclosure can be administered on multiple occasions. Intervals between single dosages can be daily, weekly, monthly or yearly. Intervals can also be irregular as indicated by measuring blood levels of modified polypeptide or antigen in the patient. Alternatively, the antigen-binding molecule can be administered as a sustained release formulation, in which case less frequent administration is required. Dosage and frequency vary depending on the half-life of the polypeptide in the patient.

It may be advantageous to formulate compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutically acceptable carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Dosages and therapeutic regimens of the antigen-binding molecule can be determined by a skilled artisan. In certain embodiments, the antigen-binding molecule is administered by injection (e.g., subcutaneously or intravenously) at a dose of about 0.01 to 40 mg/kg, e.g., to 0.1 mg/kg, e.g., about 0.1 to 1 mg/kg, about 1 to 5 mg/kg, about 5 to 25 mg/kg, about 10 to 40 mg/kg. In one embodiment, the antigen-binding molecule or chimeric molecule is administered at a dose from about 5 to 15 mg/kg, about 5 to 14 mg/kg, about 5 to 13 mg/kg, about 5 to 12 mg/kg, about 5 to 11 mg/kg or about 5 to 10 mg/kg. In one embodiment, the antigen-binding molecule or chimeric molecule is administered at a dose of about 5 mg/kg, 6 mg/kg, 7mg/kg, 8 mg/kg, 9 mg/kg, 10 mg/kg, 1 1 mg/kg, 12 mg/kg, 13 mg/kg, 14 mg/kg or 15 mg/kg. The dosing schedule can vary from e.g., once a week to once every 2, 3, or 4 weeks.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Disclosed herein is an antigen-binding molecule as defined herein, a chimeric molecule as defined herein or a pharmaceutical composition as defined herein for use as a medicament.

Disclosed herein is a method for reducing or inhibiting proliferation and/or viability of a cancer cell, the method comprising contacting the cancer cell with a therapeutically effective amount of an antigen-binding molecule as defined herein, a chimeric molecule as defined herein or a pharmaceutical composition as defined herein, wherein the cancer cell is a cancer cell selected from a mast cell tumor, a carcinoma, a sarcoma, a Leydig cell tumor or a seminoma.

The term “tumor,” as used herein, refers to any neoplastic cell growth and proliferation, whether malignant or benign, and all pre-cancerous and cancerous cells and tissues. The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. The term “precancerous” refers to a condition or a growth that typically precedes or develops into a cancer. By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer. By “early stage cancer” is meant a cancer that is not invasive or metastatic or is classified as a Stage 0, I, or II cancer. The term “late stage cancer” generally refers to a Stage III or Stage IV cancer, but can also refer to a Stage II cancer or a substage of a Stage II cancer. One skilled in the art will appreciate that the classification of a Stage II cancer as either an early stage cancer or a late stage cancer depends on the particular type of cancer. Illustrative examples of cancer include, but are not limited to, breast cancer, prostate cancer, ovarian cancer, cervical cancer, pancreatic cancer, colorectal cancer, lung cancer, hepatocellular cancer, gastric cancer, liver cancer, bladder cancer, cancer of the urinary tract, thyroid cancer, renal cancer, carcinoma, melanoma, brain cancer, non-small cell lung cancer, squamous cell cancer of the head and neck, endometrial cancer, multiple myeloma, rectal cancer, and esophageal cancer.

In one embodiment, the cancer is selected from a mast cell tumor, a mammary carcinoma, a hepatocellular carcinoma, a urothelial carcinoma, a histiocytic sarcoma, a Leydig cell tumor or a seminoma.

Disclosed herein is a method for inhibiting proliferation of a mast cell tumor, the method comprising contacting the mast cell tumor with a therapeutically effective amount of an antigen-binding molecule as defined herein, a chimeric molecule as defined herein or a pharmaceutical composition as defined herein.

Disclosed herein is a method of reducing or inhibiting proliferation, survival and/or viability of a cancer in a subject, the method comprising administering a therapeutically effective amount of an antigen-binding molecule as defined herein, a chimeric molecule as defined herein or a pharmaceutical composition as defined herein to the subject, wherein the cancer is selected from a mast cell tumor, a mammary carcinoma, a hepatocellular carcinoma, a urothelial carcinoma, a histiocytic sarcoma, a Leydig cell tumor or a seminoma.

Disclosed herein is a method of reducing or inhibiting proliferation, survival and viability of a mast cell tumor in a subject, the method comprising administering a therapeutically effective amount of an antigen-binding molecule as defined herein, a chimeric molecule as defined herein or a pharmaceutical composition as defined herein to the subject.

In one embodiment, the subject is a canine subject. In another embodiment, the subject is a feline subject.

Disclosed herein is a method of treating a cancer in a subject, the method comprising administering a therapeutically effective amount of an antigen-binding molecule as defined herein, a chimeric molecule as defined herein or a pharmaceutical composition as defined herein to the subject, wherein the cancer is selected from a mast cell tumor, a mammary carcinoma, a hepatocellular carcinoma, a urothelial carcinoma, a histiocytic sarcoma, a Leydig cell tumor or a seminoma.

Disclosed herein is a method of treating a mast cell tumor in a subject, the method comprising administering a therapeutically effective amount of an antigen-binding molecule as defined herein, a chimeric molecule defined herein or a pharmaceutical composition as defined herein to the subject.

Disclosed herein is a method of treating a disease or condition associated with an undesired expression of MYH9 in a subject, wherein the method comprises administering a therapeutically effective amount of an antigen-binding molecule as defined herein, a chimeric molecule defined herein or a pharmaceutical composition as defined herein to the subject.

In one embodiment, the disease or condition associated with an undesired expression of MYH9 is cancer. The cancer may be selected from a mast cell tumor, a mammary carcinoma, a hepatocellular carcinoma, a urothelial carcinoma, a histiocytic sarcoma, a Leydig cell tumor or a seminoma.

The term “treating” as used herein may refer to (1) delaying the appearance of one or more symptoms of the condition; (2) inhibiting the development of the condition or one or more symptoms of the condition; (3) relieving the condition, i.e., causing regression of the condition or at least one or more symptoms of the condition; and/or (4) causing a decrease in the severity of the condition or of one or more symptoms of the condition.

The terms “subject”, “patient”, “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such as from the genus Macaca (e.g., cynomolgus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. In another embodiment, the subject is a canine subject. In one embodiment, the subject is a feline subject.

The methods as disclosed herein may comprises the administration of a “therapeutically effective amount” of an agent (e.g. an antigen-binding molecule, a chimeric molecule, a polynucleotide, a construct, a vector, a host cell or a pharmaceutical composition) to a subject. As used herein the term “therapeutically effective amount” includes within its meaning a non-toxic but sufficient amount of an agent or compound to provide the desired therapeutic effect. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

In one embodiment, there is provided an antigen-binding molecule as defined herein, a chimeric molecule as defined herein or a pharmaceutical composition as defined herein for use in reducing or inhibiting proliferation, survival and viability of a cancer in a subject, wherein the cancer is selected from a mast cell tumor, a carcinoma, a sarcoma, a Leydig cell tumor or a seminoma.

In one embodiment, there is provided an antigen-binding molecule as defined herein, a chimeric molecule as defined herein or a pharmaceutical composition as defined herein for use in reducing or inhibiting proliferation, survival and viability of a mast cell tumor in a subject.

In one embodiment, there is provided a use of an antigen-binding molecule as defined herein, a chimeric molecule as defined herein or a pharmaceutical composition as defined herein in the manufacture of a medicament for reducing or inhibiting proliferation, survival and viability of a cancer in a subject, wherein the cancer is selected from a mast cell tumor, a carcinoma, a sarcoma, a Leydig cell tumor or a seminoma.

In one embodiment, there is provided a use of an antigen-binding molecule as defined herein, a chimeric molecule as defined herein or a pharmaceutical composition as defined herein in the manufacture of a medicament for reducing or inhibiting proliferation, survival and viability of a mast cell tumor in a subject.

In one embodiment, there is provided an antigen-binding molecule as defined herein, a chimeric molecule defined herein or a pharmaceutical composition as defined herein for use in treating a cancer in a subject, wherein the cancer is selected from a mast cell tumor, a carcinoma, a sarcoma, a Leydig cell tumor or a seminoma.

In one embodiment, there is provided an antigen-binding molecule as defined herein, a chimeric molecule defined herein or a pharmaceutical composition as defined herein for use in treating a mast cell tumor in a subject.

In one embodiment, there is provided the use of an antigen-binding molecule as defined herein, a chimeric molecule defined herein or a pharmaceutical composition as defined herein in the manufacture of a medicament for treating a cancer in a subject, wherein the cancer is selected from a mast cell tumor, a carcinoma, a sarcoma, a Leydig cell tumor or a seminoma.

In one embodiment, there is provided the use of an antigen-binding molecule as defined herein, a chimeric molecule defined herein or a pharmaceutical composition as defined herein in the manufacture of a medicament for treating a mast cell tumor in a subject.

In one embodiment, there is provided an antigen-binding molecule as defined herein, a chimeric molecule defined herein or a pharmaceutical composition as defined herein for use in treating a disease or condition associated with an undesired expression of MYH9 in a subject.

In one embodiment, there is provided the use of an antigen-binding molecule as defined herein, a chimeric molecule defined herein or a pharmaceutical composition as defined herein in the manufacture of a medicament for treating a disease or condition associated with an undesired expression of MYH9 in a subject.

Disclosed herein is a method of detecting the likelihood of the presence of a cancer in a subject, the method comprising determining the level of MYH9 in a sample obtained from the subject, wherein an increased level of MYH9 as compared to a reference indicates the likelihood of the presence of a cancer in a subject, wherein the cancer is selected from a mast cell tumor, a carcinoma, a sarcoma, a Leydig cell tumor or a seminoma.

Disclosed herein is a method of detecting the likelihood of the presence of a mast cell tumor in a subject, the method comprising determining the level of MYH9 in a sample obtained from the subject, wherein an increased level of MYH9 as compared to a reference indicates the likelihood of the presence of a mast cell tumor in a subject.

In one embodiment, the sample is a cell, tissue or blood sample.

In one embodiment, the method comprises contacting the sample with an antigen-binding molecule as defined herein or a chimeric molecule as defined herein to determine the level of MYH9 in the sample.

Disclosed herein is a method for predicting the prognosis of a cancer in a subject, the method comprising determining the level of MYH9 in a sample obtained from the subject, wherein an increased level of MYH9 as compared to a reference indicates a likelihood of a poor prognosis associated with tumor invasiveness in the subject, wherein the cancer is selected from a mast cell tumor, a carcinoma, a sarcoma, a Leydig cell tumor or a seminoma.

Disclosed herein is a method for predicting the prognosis of a mast cell tumor in a subject, the method comprising determining the level of MYH9 in a sample obtained from the subject, wherein an increased level of MYH9 as compared to a reference indicates a likelihood of a poor prognosis associated with tumor invasiveness in the subject.

In one embodiment, the poor prognosis is associated with poor survival in the subject.

The present disclosure also extends to a kit comprising the antigen-binding molecule, or the vector, or the pharmaceutical composition, as described herein.

Also disclosed herein is the use of the antigen-binding molecule, or the vector, as described herein, for detecting MYH9 in a sample.

By “about” is meant a quantity, level, value, number, frequency, percentage, dimension, size, amount, weight or length that varies by as much 15, 14, 13, 12, 11, 10, 9, 8, 7, 6, 5, 4, 3, 2 or 1% to a reference quantity, level, value, number, frequency, percentage, dimension, size, amount, weight or length.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### EXAMPLES

In our study, we have developed a therapeutic antibody-drug conjugate (cIgG_{B}_12B6FcX-MMAE) for the treatment of cMCTs in dogs. In in-vitro studies, it demonstrate specific reactivity towards malignant mast cell phenotype and is capable of inducing cell death by delivering MMAE toxin into reactive cells. Its cytotoxic ability is also observed in in-vivo xenograft mouse model where complete tumor remission is observed in mice treated with cIgG_{B}12B6FcX-MMAE. With its specific cytotoxicity, cIgG_{B}12B6FcX-MMAE is a promising therapeutic candidate for the treatment of cMCTs.

### Materials and Methods

### Culture

Canine cancer cell lines and their proprietary culture media were in-kind contributions from Canfel Therapeutics, USA. MDCK (CCL-34) and Cf2Th (CRL-1430) cell lines were purchased from American Type Culture Collection (ATCC, USA) and cultured according to ATCC's recommendations. All cell lines were maintained at 37° C. in a humidified incubator with 5% CO₂.

### Antibody Generation

mIgG_12B6 was generated by a mouse hybridoma approach using live intact cMCT01 cells as the immunogen. Hybridomas were maintained in ClonaCell-HY Medium E (Stem Cells Technologies) at 37° C. in a humidified incubator with 5% CO₂.

### Isotyping

Isotyping was performed with Mouse Monoclonal Antibody Isotyping kit from Roche (Roche, #11493027001) according to manufacturer's instructions. Briefly, the pellet in the tube was reconstituted with 150 μl of hybridoma culture supernatant. The solution was thoroughly mixed by vortexing before adding the isostrip. The results were analyzed after 10 min of incubation.

### Protein Lysate Extraction

Suspension cMCT01 cells were pelleted down at 1500 rpm for 5 minutes before washing with ice cold PBS (Invitrogen, USA). The pellet was resuspended in lysis buffer consisting of 2% Triton in PBS supplemented with protease Inhibitor cocktail (Calbiochem-Novabiochem, UK) at 1:100 dilution and left on ice for 15 minutes. The mixture obtained was subsequently transferred to a 1.5mL microcentrifuge tube and was centrifuged at 14,000 g for 1 minute at 4° C. The supernatant was collected as cell lysate and transferred to a new tube.

### Protein Quantification using DC Assay

Protein standards were prepared with BSA concentrations ranging from 0.2 mg/mL to 1.5 mg/mL. Lysate samples were prepared at 1:5 and 1:10 dilutions with 2% Triton/PBS. 5 μL of standards and samples were then added into microwells, followed by 25 μL of Reagent A′ and 200 μL of reagent B. Reagent A′ was prepared by adding reagent S (BioRad, USA) to reagent A (BioRad, USA) at 1:50 dilution. Subsequently, the plate was incubated in the dark at room temperature for 15 minutes. The absorbance values of standards and samples at 650nm were read using Tecan I-control (Tecan, Switzerland) and the quantities of protein in the samples were determined using the standard curve generated.

### Immunoprecipitation

cMCT01 cells were subjected to protein lysate extraction and the cell lysate collected was used immediately for immunoprecipitation (IP). IP was carried out using the automated Phynexus MEA system (Phynexus, Inc., USA). Briefly, the mIgG_12B6 was directly captured onto Protein G PhyTip columns. After washing away unbound proteins with Wash Buffer I (10 mM NaH₂PO₄/140 mM NaCl pH 7.4), clarified cell lysate was then passed through the column functionalized with mIgG_12B6. The column was further washed with Wash Buffer II (140 mM NaCl pH 7.4), and bound proteins were eluted at low pH with Elution Buffer (200 mM Na H₂PO₄/140 mM NaCl pH 2.5). The eluate was neutralized immediately with 1 M Tris-Cl pH 9.0 and subsequently resolved by SDS-PAGE.

### Protein Gel Electrophoresis

Samples were prepared with loading dye at a final concentration of 2% SDS, 10% glycerol and 0.02% bromophenol blue. For reducing conditions, 2% mercaptoethanol was added. Subsequently, the samples were heated for 5 minutes at 95° C. before resolving on a 4-12% Bis Tris gel (Thermo Fisher Scientific) with 1×MOPS running buffer (Thermo Fisher Scientific) at 150V for 2 hours. The final gel was either stained using Coomassie Brilliant Blue staining solution (Sigma Aldrich) or transferred to PVDF membrane (BioRad, USA) via western blotting.

### Western Blot

The resolved proteins from the gel run were transferred onto PVDF membrane with transfer buffer consisting of 20% methanol, 25 mM Tris and 192 mM glycine at 110V for 2 hours. Subsequently, the membrane was incubated with 5% non-fat milk for 1 hour. After blocking, the membrane was incubated overnight at 4° C. with primary antibody (1:1000 purified mIgG_12B6; 1:1000 rabbit anti-MYH9 pAb (Thermo Fisher Scientific); 1:1000 mouse anti-MYH9 mAb (Abcam). After multiple washes with buffer consisting of 0.1% Tween-20/PBS, the membrane was incubated with appropriate HRP-conjugated secondary antibody for 1 hour and visualized by addition of chemiluminiscence substrate (GE Healthcare). Blot images were either developed on Lumi-film (Roche) using Medical X-ray Processor 2000 (Kodak) or captured using ChemiDoc Imaging System (BioRad).

### Coomassie Staining and Liquid Chromatography-Mass Spectrometry

The gel was immersed in staining solution consisting of 0.1% w/v Coomassie Brilliant Blue, 10% acetic acid, 50% methanol for 30 minutes, followed by overnight destaining with 10% acetic acid, 50% methanol in water. Protein bands corresponding to the antigen of interest were manually excised and kept in 2.5 mM ammonium bicarbonate, 50% acetonitrile solution at 4° C. Samples were subjected to in-gel trypsin proteolysis before processing via liquid chromatography-tandem mass spectrometry.

### Knockdown of MYH9 by Small Interfering RNA (siRNA)

10⁶ cMCT01 cells were transfected with either validated siRNA targeted against Myosin-9 (Ambion, #4390824) or non-targeting siRNA negative control 3 (Ambion, #AM4615) via the SF Cell Line 4D-Nucleofection Kit (Lonza). After transfection, the cells were harvested at 48 hours for protein extraction and western blot analysis.

### Flow Cytometry Analysis

Cells were harvested as single cell suspensions and resuspended at 2×10⁵ cells in 10 μL of 1% bovine serum albumin (BSA)/PBS. Samples were incubated for 30 minutes with 5 μg of primary antibody on ice and then washed with cold 1% BSA/PBS before further incubating for 10 minutes in the dark with FITC-conjugated goat anti-mouse antibody (DAKO) at 1:500 dilution. Caninized antibodies, which were directly conjugated with fluorochrome (FITC or AlexaFluor488), were added to cells on ice and incubated for 30 minutes. After incubation, the cells were washed again and resuspended in 200 μL of 1% BSA/PBS for analysis on a MacsQuant Analyzer 10 (Miltenyi Biotec). and subsequently acquired with MacsQuant Analyzer.

### Periodate Treatment

Total protein lysate and membrane protein fractions were subjected to gel electrophoresis and transfer to PVDF membrane as described above. After the membrane was blocked for 30 minutes in 5% non-fat milk, it was rinsed twice with 10 mL of 100 mM sodium acetate at pH 4.5. Subsequently, the membrane was incubated twice with 5 mL of 100 mM sodium metaperiodate (Sigma Aldrich) for 30 minutes in the dark. After multiple washes with sodium acetate, the membrane was then incubated with 5 mL of 0.5M sodium borohydride for 30 minutes. After rinsing the membrane with PBS, it was blocked with 5% non-fat milk again before incubating with mIgG_12B6 overnight. A non-treated control was done in the absence of sodium metaperiodate.

### PNGase F Treatment

20 ug of cMCT01 cell lysate was first denatured at 100° C. and then digested with 1000U of PNGase F enzyme (New England Biolabs) for 1 hour at 37° C. The digested sample and non-treated control were further analyzed via gel electrophoresis and western blot.

### Pronase Treatment

100 ug of cMCT01 cell lysate was subjected to Pronase (Sigma Aldrich) treatment at 2 mg/ml for 1 hour at 37° C. 5 ul of digested sample and non-treated control were immobilized on PVDF membrane and subsequently subjected to western blot analysis.

### Proliferation-CellTiter-Glo Luminescent Cell Viability (CTG) Assay

Cells were seeded into a black coated 96 well plate (Grenier Bio-one) at 2000 cells per well in 90 μof media. 24 hours post-seeding, 10 μL of mIgG_12B6 drug complex (MMAE or Saporin) or buffer was added to each well. The plate was incubated at 37° C. for another 48 to 72 hours before cell viability was assessed via CTG assay (Promega) according to manufacturer's instructions.

### Antibody Drug Conjugate (ADC) with Secondary Saporin or MMAE Conjugates

For mIgG_12B6 mAb, 2 μg of antibody was pre-incubated with either MMAE conjugated anti-mouse IgG (Moradec) or Saporin conjugated anti-mouse IgG (Advanced Targeting Systems) at 1:1 molar ratio for 15 minutes in 100 ul of media. Cells were seeded as described above and 10 ul of the resultant mIgG_12B6-drug complex was added into each well at final concentration of 2 μg/ml. The viability of the treated cells were assessed 48-72 hours later via CTG assay.

Caninized mAbs were biotinylated (Pierce) and pre-incubated with streptavidin-Saporin (Advanced Targeting System) at 1:2 molar ratio for 15 minutes prior addition to cells. Viability of the treated cultures were assessed 48-72 hours later via CTG assay.

### Immunohistochemistry (IHC)

Cryosections were thawed to room temperature and briefly fixed in 10% neutral buffered formalin (NBF) for 10 minutes. Subsequently, sections were washed with PBS and blocked with 10% goat serum (DAKO), followed by primary incubation with 3-5 μg/ml of mIgG_12B6 at 4° C. overnight. The sections were then developed using the EnVision+System-HRP DAB Kit (DAKO) in the fume hood for 2-3 minutes and counterstained with hematoxylin. Sections were adequately dehydrated through 50%, 70%, 90% and 100% ethanol and mounted using Mounting Organo Mounting Medium (Sigma Aldrich).

Formalin-fixed paraffin-embedded (1-1-PE) sections were placed in 60° C. oven for 40 minutes, dewaxed in Histoclear (10 min×2) and rehydrated in a series of graded ethanols to distilled water. Antigen retrieval was performed in Tris buffer, pH 10 (Abcam) at 95° C. for 20 minutes, followed by cooling to room temperature. FFPE sections were quenched in 3% hydrogen peroxide to remove endogenous peroxidase, followed by blocking in 10% goat serum (DAKO). The slides were incubated with 30-50 ug/ml of mIgG_12B6 at 4° C. overnight and developed using the EnVision+System-HRP DAB Kit (DAKO) in the fume hood for 2-3 minutes and counterstained with hematoxylin.

### cDNA Synthesis and PCR Amplification of V_{H} and V_{L} Variable Regions

Total RNA was extracted from hybridoma cells using the NucleoSpin RNA Isolation Kit (Macherey-Nagel) as described by the manufacturer. 2 μg of total RNA was reverse transcribed (RT) with MMLV reverse transcriptase (Thermo Fisher Scientific) in reaction buffer (50 mM Tric-HCl, 75 mM KCl, 3 mM MgCl2, 10 mM DTT), 25 U RNase inhibitor, 1 mM of each dNTPs and 2 μg of Oligo(dT) primer (Thermo Fisher Scientific). The cDNA synthesis mixture was subjected to 25° C. for 5 minutes, 42° C. for 1 hour and 70° C. for 15 minutes. PCR amplification was carried out in final volume of 50 μl containing 5 μl of cDNA synthesis reaction, 200 μM dNTPs, 5 mM MgCl2, 5 U Taq Polymerase and 10 μl of 5×GoTaq Flexi reaction buffer (Promega). 1 nM of forward and reverse primers were used. The primer sequences for isolating VH and VL variable regions are as follows:

| | |
|---|---|
| VH Forward | 5′ GAC AGT GGA TAR ACM GAT GG 3′ |
| VH Reverse | 5′ GAG GTS MAR CTG CAG SAG TCW GG 3′ |
| VL Forward | 5′ GGA TAC AGT TGG TGC AGC ATC 3′ |
| VL Reverse | 5′ CAA ATT GTT CTC ACC CAG TCT 3′ |

The thermal cycle was programmed for 2 minutes at 95° C. for initial denaturation, followed by 35 cycles of 1 minute at 9 ° C. for denaturation, 1 minute at 55° C. for annealing, 1 minute at 72° C. for extension, and 5 min at 72° C. for the final extension. PCR products were examined by electrophoresis at 100 V for 30 min in a 1% (w/v) agarose gel in 1×TAE buffer and purified using QIAquick Gel Extraction Kit (Qiagen) according to manufacturer's instructions. The purified DNA product was ligated into pGEM-T vector (Promega) and transformed into DH5 competent cells (Thermo Fisher Scientific). Clones were selected from agar plates and cultured in ampicillin containing LB broth, followed by plasmid purification using QIAprep Spin Miniprep Kit (Qiagen). The plasmid inserts were subsequently sequenced using M13F and M13R primers.

### Generation of CHO Pools Expressing Chimeric cIgG_12B6

mIgG_12B6 variable and canine IgG constant region sequences were synthesized and cloned into the pTarget2.2-Hum expression vector (Genscript). cIgG_{A}_12B6 and cIgG_{B}_12B6 were expressed in DG44-CHO cells and purified using Protein G Sepharose medium (GE Healthcare).

### Expression of MALA-PG Variant using pTarget2.2-Hum-cIgG_{B}_12B6

Chimeric cIgG_{B}_12B6FcX variant was generated by performing site mutations at M₂₃₈A, L₂₃₉A and P₃₃₃G on canine IgG_{B} heavy constant region of pTarget2.2-Hum-cIgGB_12B6 vector. The variant was expressed in CHO cells and purified using Protein G Sepharose medium (GE Healthcare).

### Dose Response Curve (IC₅₀)

Direct conjugation of MMAE to cIgG _{B}_12B6FcX was done by Moradec with drug to F(ab′)₂ ratio of 4.0. Cytotoxicity of cIgG_{B}_12B6FcX-MMAE was evaluated on cMCT01 cell line. Cells were seeded in 96-well plate at 2000 cells per well. cIgG_{B}_12B6FcX-MMAE was serially diluted and added to the cells at final concentration ranging from 100 nM to 0.0001 nM. The viability of the treated cells was evaluated after 72 hours using the CTG assay kit (Promega). IC₅₀ values were calculated with GraphPad Prism 6 Software (GraphPad).

### In-Vivo Efficacy of mIgG 12B6-SAP in Pre-Emptive cMCT01 Xenograft Model

Female athymic nude mice (˜6 weeks) were subcutaneously injected with 5×10⁶ cMCT01 cells resuspended in 50% high concentration Matrigel matrix (Corning). Mice were randomly separated into groups (n=5 per group) and treatments were initiated immediately after inoculation. Mice were administered with 37.5 ug of mIgG_12B6-SAP (ATS) weekly for 3 weeks via intraperitoneal injection. Tumor size was measured weekly using digital calipers and tumor volume calculated using (LxW²)/2, where L is the longest and W is the shortest in tumor diameters (mm).

### In-Vivo Efficacy of cIgG_{B}_12B6FcX-MMAE in Established cMCT01 Xenograft Model

5×10⁶ cMCT01 cells were resuspended in 50% high concentration Matrigel matrix (Corning) and subcutaneously injected into female athymic nude mice (˜6 weeks). They were randomly grouped under 6 conditions (n=4-5 per group) when tumor volume reached 200 mm³. Treatment was administered once every 4 days via intravenous tail injection for 3 doses. The mice were monitored twice a week for experimental duration of 78 days. Tumor volume was calculated using (LxW²)/2, where L is the longest and W is the shortest in tumor diameters (mm).

### Example 1

### Results

### mIgG_12B6 Binds Specifically to Canine Mast Cell Lines and Mast Tumor Tissue

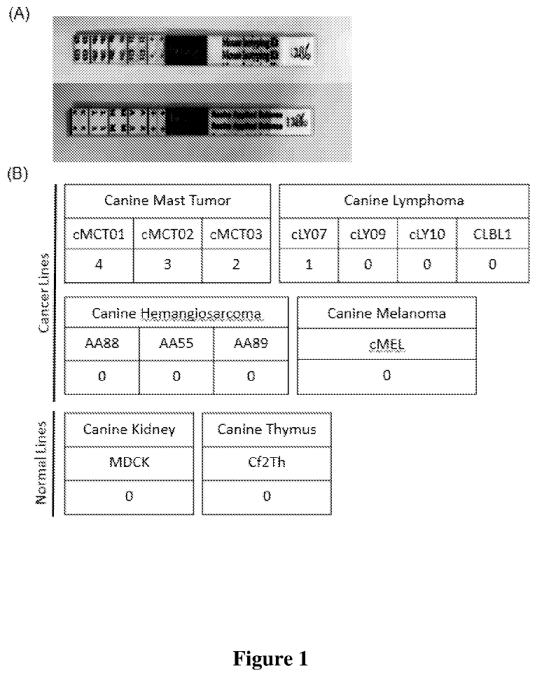

mIgG_12B6 was generated in Balb/c mice using whole mast cancer cell (cMCT01) as immunogen. It was isotyped to be mouse IgG1 (FIG. 1A). Flow cytometry analysis was subsequently performed on different canine cancer and normal cell lines to assess cell surface reactivity with mIgG_12B6 (FIG. 1B). mIgG_12B6 was found to bind to all 3 canine mast cancer cell lines but not on normal canine cell lines Minimal to negative binding was also observed also on other types of canine cancer cell lines such as lymphoma, hemangiosarcoma and melanoma.

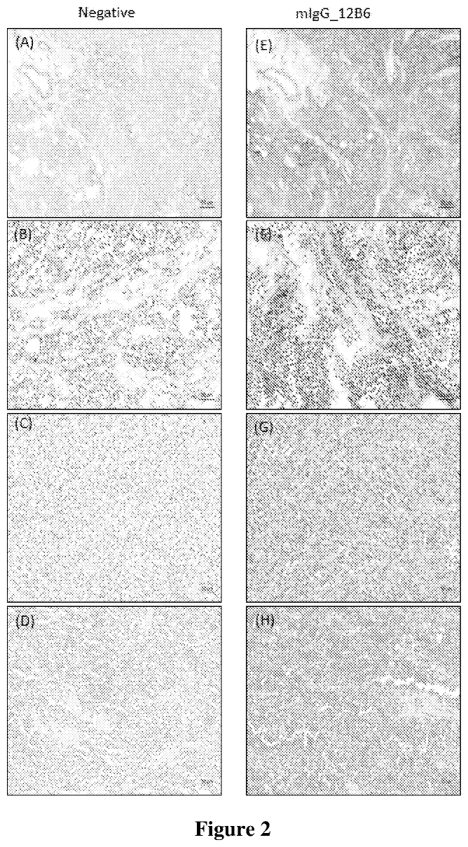

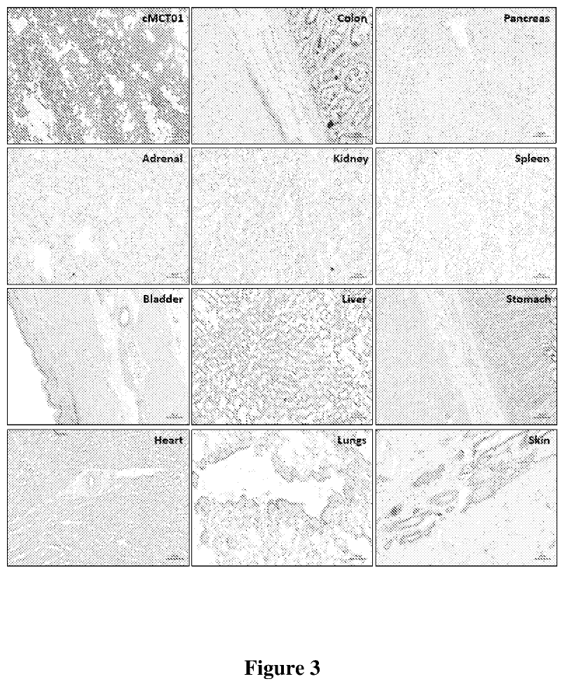

To evaluate whether canine cell lines and tissues share similar reactivity, IHC staining was performed on canine mast tumour and normal tissue cryosections. Mast tumour tissues were significantly stained with mIgG_12B6 antibody in comparison to its negative control (FIG. 2), whereas little or no antibody staining was observed for normal canine tissues (FIG. 3). These observations suggested specificity of mIgG_12B6 reactivity to malignant mast phenotype.

### mIgG_12B6 Targets Canine Myosin-9 (MYH-9)

mIgG_12B6 antigen was initially detected on western blot as a single 140 kDa protein band under both reducing and non-reducing conditions (FIG. 4A). Subsequently, we proceeded with antigen identification via immunoprecipitation (IP) (FIG. 4B) to enrich for the antigen of interest from cMCT01 cell lysate using Protein G tips functionalized with mIgG_12B6 antibody. As seen in FIG. 4B, a protein band of same molecular weight as the antigen was observed in the IP lane, but absent in mouse IgG1 isotype control and mIgG_12B6 antibody control lanes. This suggested that the antigen of interest had been specifically enriched. The equivalent antigen band from a parallel ran SDS-PAGE gel was excised and sent for mass spectrometry analysis for protein identification. The antigen was identified as Myosin-9 (MYH-9, Accession #Q258K2). Peptide sequences, identified by several rounds of mass spectrometry, accounted for approximately 40% of the full length protein (FIG. 4C). To validate the identity of the antigen, a commercially available anti-MYH-9 was used to immunoblot against mIgG_12B6 IP eluate. As shown in FIG. 5A, commercial anti-MYH9 was able to detect the antigen band at 140 kDa in the IP lane, hence confirming the identity of the antigen as MYH-9. In addition, transient knockdown of MYH-9 expression was performed on cMCT01 cells using targeted siRNA. In MYH-9 siRNA-treated sample (FIG. 5B, KD lane), MYH-9 protein expression was significantly reduced compared to non-treated (FIG. 5B, SF lane) or non-targeting siRNA-treated (FIG. 5B, SC lane) samples. Similarly, mIgG_12B6 antigen detection was also lowered in MYH-9 siRNA-treated samples, hence validating the identity of mIgG_12B6 antigen as MYH-9.

### mIgG_12B6 Binds to Glycoprotein Epitope

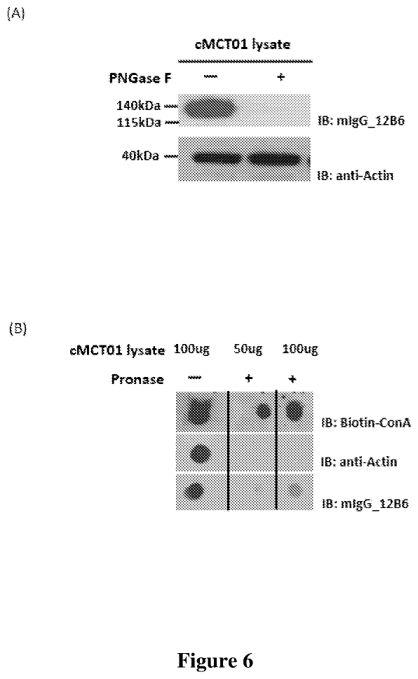

After removal of N-linked glycans from cMCT01 lysate with PNGase F, antigen recognition by mIgG_12B6 was completely lost, implying that N-linked glycans are necessary for antigen reactivity (FIG. 6A). In contrast, there is no loss in reactivity for anti-actin antibody, which bound solely to the actin protein.

Subsequently, cMCT01 lysate was subjected to pronase treatment, which digested proteins into amino acids. A loss in antigen reactivity by mIgG_12B6 is observed after pronase digestion (FIG. 6B), suggesting that intact protein structure is also necessary for mIgG_12B6 reactivity.

These observations confirmed the binding epitope of mIgG_12B6 as a glycopeptide.

### mIgG_12B6 Acts as Vehicle for Targeted Toxin Delivery

To assess the potential of mIgG_12B6 as a targeting agent for toxin delivery, an in-vitro assay was performed by incubating mast tumor cell cultures with mIgG_12B6-toxin complex. The antibody was pre-incubated with secondary toxin conjugates such as anti-mouse IgG saporin (SAP) or anti-mouse IgG auristatin (MMAE) to form mIgG_12B6-toxin complex. The cytotoxicity of mIgG_12B6-toxin complex in mast tumor (cMCT01) and hemangiosarcoma (AA88) cell cultures were then evaluated via CellTitre Glo (CTG) assay at 48 hours post treatment.

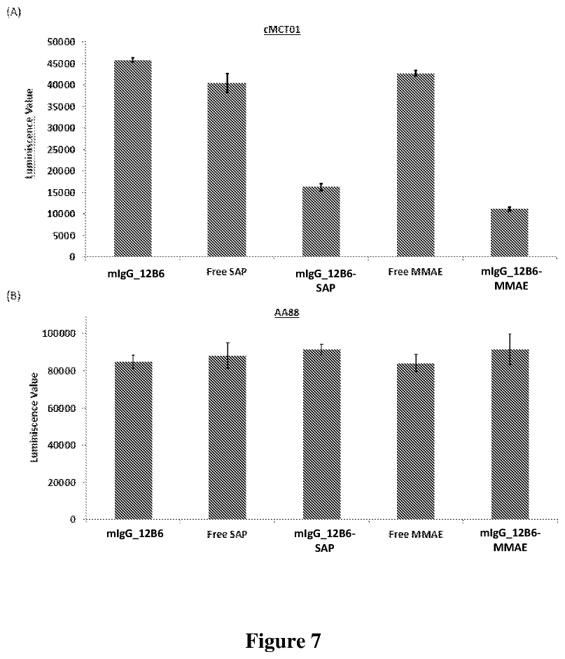

As shown in FIG. 7A, mIgG_12B6-SAP and mIgG_12B6-MMAE complex were able to exert a potent cytotoxic effect on cMCT01 cell line, lowering their viabilities to 33% and 27% respectively. Conversely, both mIgG_12B6-SAP and mIgG_12B6-MMAE exhibited no cytotoxic effect against AA88, a non-binding cell line (FIG. 7B). As a control, the cell lines were treated with free toxins and no cytotoxicity were observed. These data demonstrated effective internalization and targeted in-vitro cytotoxicity of mIgG_12B6-toxin complex and mIgG_12B6 can indeed serve as a vehicle for targeted delivery of conjugated toxin.

### mIgG_12B6-SAP Exhibits Potent Anti-Tumor Activity in Mouse Xenograft Model

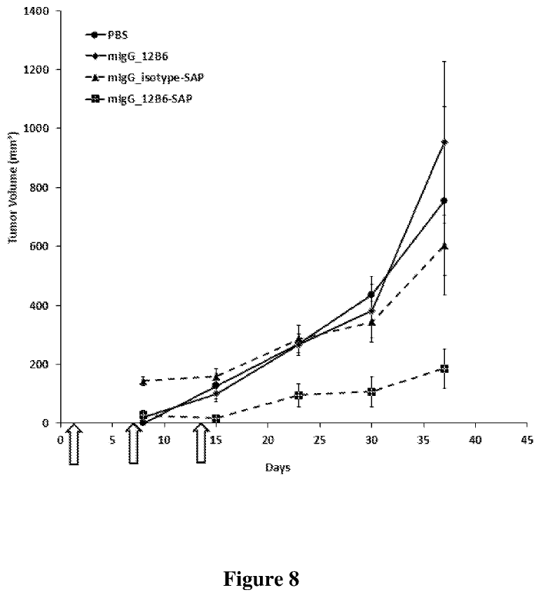

To assess in-vivo efficacy of mIgG_12B6-SAP in a pre-emptive xenograft model, NCr nude mice injected with cMCT01 cells were treated with mIgG_12B6-SAP for 3 weeks at dose of 1.875 mg/kg. As shown in FIG. 8, tumor growth of mIgG_12B6-SAP treated mice was significantly inhibited as compared to PBS and 4D01-SAP control groups. As measured on day 35, the average tumor volume of mIgG_12B6-SAP mice was 75% smaller than that of PBS treated mice.

### Characterization of Chimeric Canine cIgG_{A}12B6 and cIgG_{B}12B6

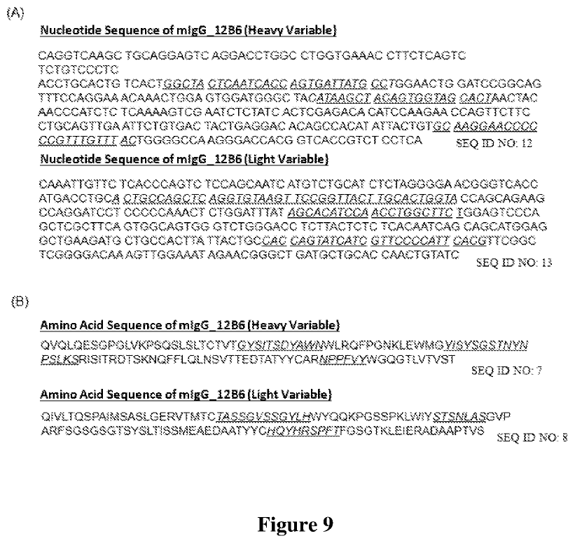

cDNA encoding for mIgG_12B6 variable domains were successfully cloned via RT-PCR and then sequenced to identify the complementarity-determining regions (CDR). The nucleotide and amino acid sequences were shown in FIG. 9A and FIG. 9B with their respective CDR regions underlined.

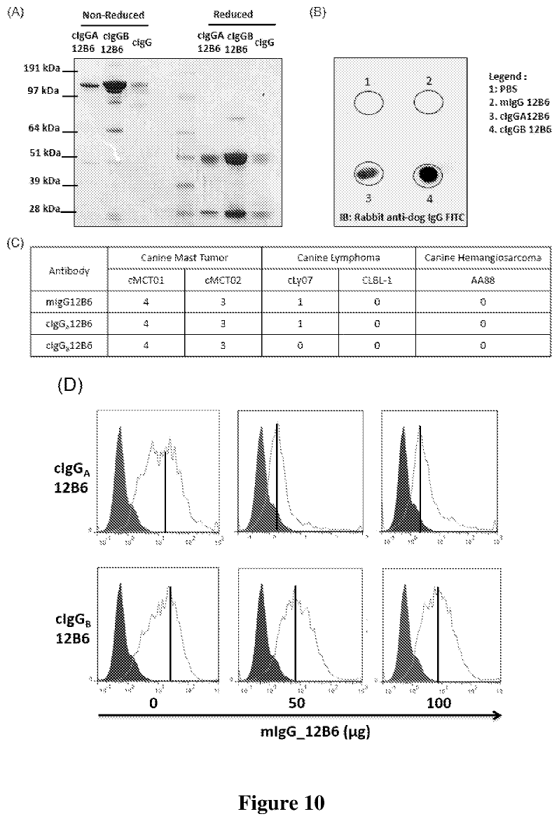

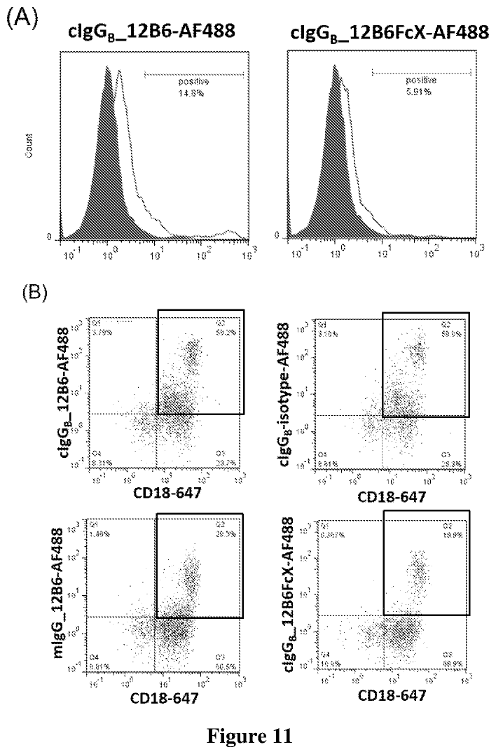

Subsequently, mIgG_12B6 variable domain sequences were synthesized and cloned into expression vector containing canine IgG_{A} or IgG_{B} constant genes. The final vectors were transfected into CHO cell line for stable expression of chimeric canine 12B6 (cIgGA_12B6 and cIgGB_12B6) antibodies. These antibodies were purified using Protein G resin and resolved on the SDS-PAGE gel. As shown in FIG. 10A, both antibodies were observed at 150 kDa under non-reducing conditions, which is the correct molecular weight for intact whole IgG. For reducing conditions, bands at 50 kDa and 25 kDa were also observed for both antibodies, which accounts for the heavy and light chains of the IgG molecule.

To demonstrate effective chimerization, a dot blot was performed to assess reactivity of chimeric canine 12B6 antibodies to anti-dog secondary conjugate (FIG. 10B). Positive signals were detected for both cIgG_{A}_12B6 and cIgG_{B}_12B6 dots when immunoblotted with rabbit anti-dog FITC as primary antibody. No signal was detected for mIgG_12B6 and PBS control. These results confirm effective chimerization of mIgG_12B6 to chimeric cIgG_{A}_12B6 and cIgG_{B}_12B6.

Next, the binding profile of cIgG_{A}_12B6 and cIgG_{B}_12B6 with canine cancer cell lines was evaluated via flow cytometry analysis (FIG. 10C). Chimeric canine 12B6 antibodies were found to bind to canine mast tumor cell lines in a similar profile as its parental mIgG_12B6. Furthermore, no binding was also detected for cell lines such as AA88 and CLBL-1, which were non-reactive to mIgG_12B6. These results suggested that chimerization did not alter its specific interaction with canine mast tumor cells.

To further investigate antigen-antibody reactivity, we pre-incubated cMCT01 cells with parental mIgG_12B6 before subjecting the cells to AF488 conjugated chimeric 12B6 antibodies. A decrease in binding with the pre-incubated cells was observed when compared with non-incubated cells, suggesting that mIgG_12B6 was able to block and bind competitively with its chimeric equivalent (FIG. 10D). Taken together, these results indicated conservation of binding epitope between mIgG_12B6 and its chimeric 12B6 antibodies.

### MALA PG variant of cIgG_{B}_12B6 has Lower Fc Binding

To mitigate FcR reactivity, a variant of cIgG_{B}_12B6, cIgG_{B}_12B6FcX, was expressed with 3 amino acid mutations on the Fc region. Flow cytometry analysis of cIgG_{B}_12B6FcX on canine PBMCs and normal myeloid population (CD18 positive) showed decreased reactivity compared to cIgG_{B}_12B6. As shown in FIG. 11B, the binding population for CD18 positive cells was lowered to 19.9% after Fc mutation. These results indicated effective reduction in binding with FcR expressing canine cells for cIgG_{B}_12B6FcX.

### Chimeric Canine cIgG_{b}_12B6FcX Toxin Complex Induces Cytotoxicity In-Vitro

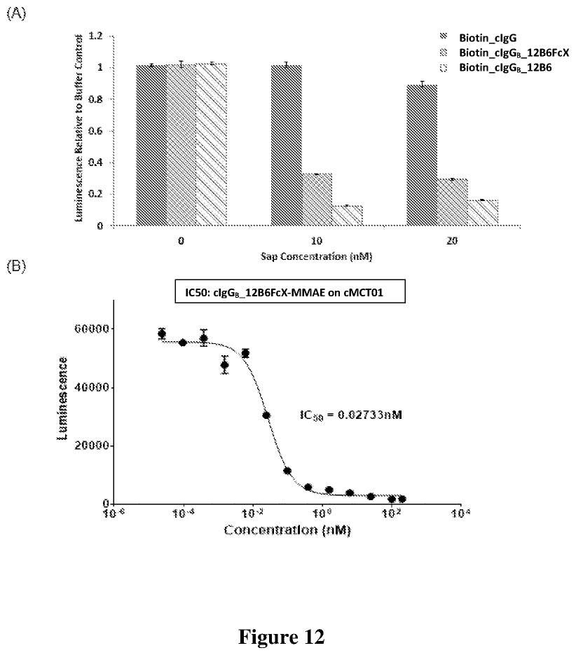

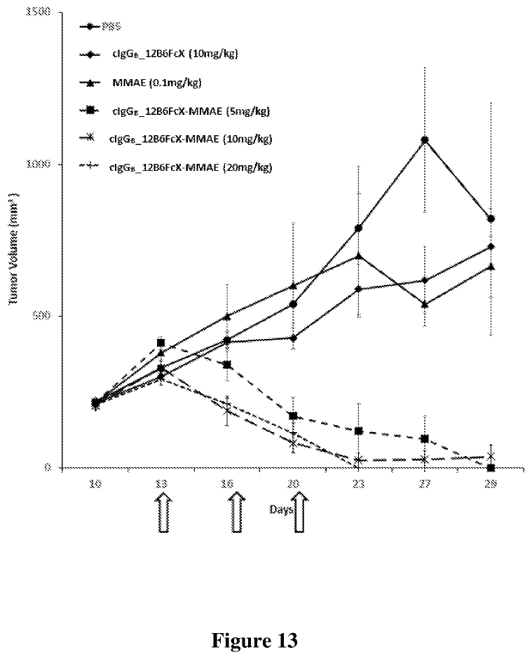

Subsequently, the cIgG_{B}_12B6FcX was assessed for its internalization ability. Biotinylated cIgG_{B}_12B6FcX and cIgG_{B}_12B6 antibodies were indirectly conjugated to streptavidin-saporin at molar ratio of 1:2 to form cIgG_12B6-saporin complex. As an isotype control, canine IgG was similarly conjugated. Increasing concentrations of both complexes were added to cMCT01 cultures and viabilities assessed 72 hours post treatment via CTG assay. As shown in FIG. 12A, increasing cytotoxicity was observed in dose-dependent manner in cIgG_12B6-saporin treated cultures, indicating an effective internalization of toxin resulting in cell death.

cIgG_{B}_12B6FcX-MMAE, synthesized by direct conjugation of MMAE to cIgG_{B}_12B6FcX, was added to cMCT01 cultures at concentrations ranging from 10⁻⁴ nM to 10² nM. IC₅₀ values for cIgG_{B}_12B6FcX-MMAE on cMCT01 were calculated to be 0.02733 nM (FIG. 12B).

### cIgG_{B}_12B6FcX-MMAE Exhibits Potent Anti-Tumor Activity in cMCT01 Xenograft Model

To assess in-vivo efficacy of cIgG_{B}_12B6FcX-MMAE in an established xenograft model, NCr nude mice injected with cMCT01 cells were treated with 3 doses of cIgG_{B}_12B6FcX-MMAE_at 5.0 mg/kg, 10 mg/kg and 20 mg/kg. As shown in FIG. 13, tumor size of cIgG_{B}_12B6FcX-MMAE treated mice was significantly reduced and complete remission was observed on day 29. Mice treated with 10 mg/kg and 20 mg/kg of cIgG_{B}_12B6FcX-MMAE remained in complete remission for the rest of the experimental duration, while 1 out of 4 mice treated with 5.0 mg/kg of cIgG_{B}_12B6FcX-MMAE had a tumor relapse at day 38.

### Prevalence of mIgG_12B6 Reactivity on Canine Tumor Tissue Microarray (cTMA)

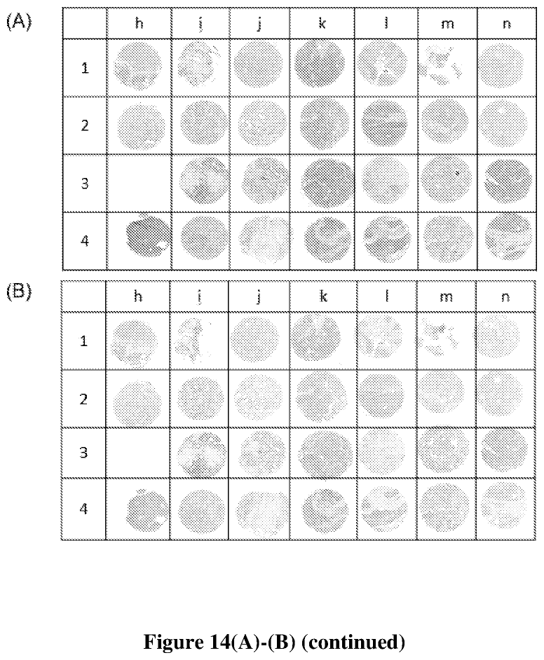

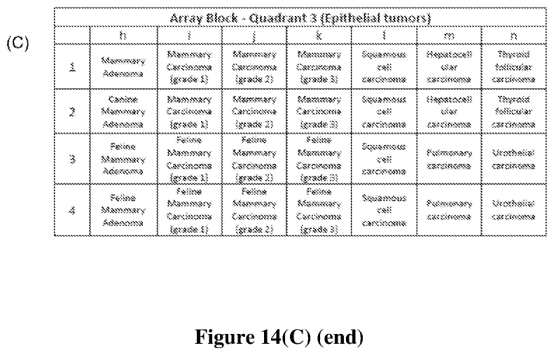

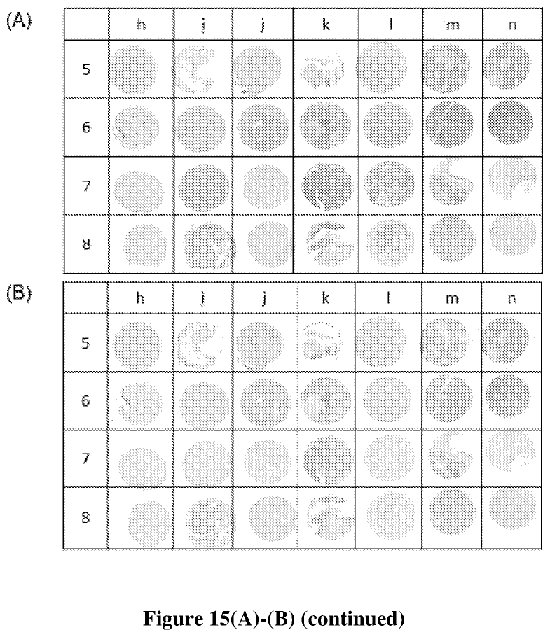

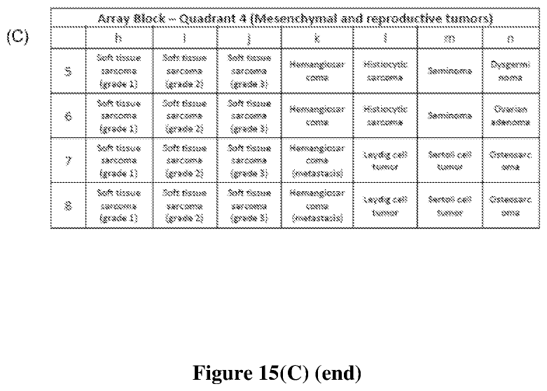

To evaluate the prevalence of mIgG_12B6 reactivity in various canine tumor tissues, we performed IHC staining on tissue microarray consisting of canine/feline epithelial tumor (FIG. 14), canine mesenchymal and reproductive tumors (FIG. 15). Positive mIgG_12B6 staining was observed in cancer indications such as feline and canine mammary carcinoma (k1-k4), canine hepatocellular carcinoma (m1-m2), canine urothelial carcinoma (n3-n4), canine histiocytic sarcoma (15-16), Leydig cell tumor (17-18) and canine seminoma (m5-m6). This indicates the possibility of extending the therapeutic ability of mIgG_12B6 as an ADC to these tumor indications.

### Preclinical Single-Dose Toxicology Study of cIgG_{B}_12B6FcX-MMAE in Mice

To evaluate the tolerable limits and determine adverse effects of cIgG_{B}_12B6FcX-MMAE in vivo, ICR mice (2 male and 2 female) were treated with a single dose of cIgG_{B}_12B6FcX-MMAE at 5 mg/kg, 10 mg/kg and 15 mg/kg. The mice were monitored for mortality, clinical signs, body weights, and food consumption for 9 days. Upon completion of 9-day observation period, blood samples were collected and subjected to hematology analysis (complete blood count with white blood cell differential) and clinical chemistry analysis (renal and liver profile). Subsequently, all animals were euthanized and subjected to a complete necropsy. Selected tissues (adrenal gland, brain, esophagus, heart, kidney, liver, lung with large bronchi, ovary/testis, pancreas, spleen, thymus, thyroid) were collected from the mice and evaluated by histopathological examination.

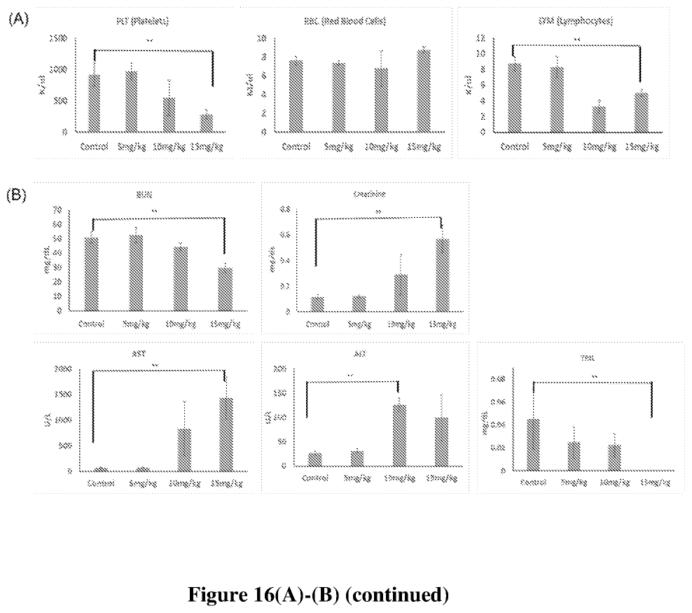

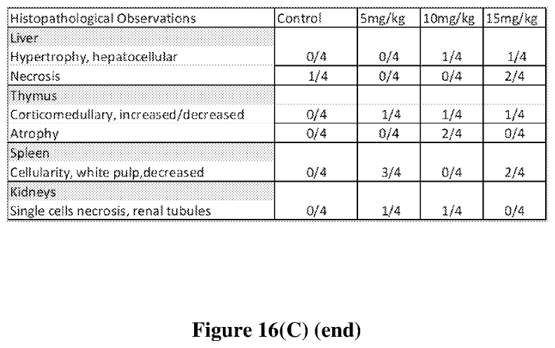

There were no significant behavioral abnormalities, weight loss or mortality observed during the study. Similarly, no adverse pathological observations were made during the necropsy procedure. However, the changes in clinical chemistry parameters (FIG. 16B) and hematology blood count (FIG. 16A) provided some evidence for potential toxicity. The significant changes in the levels of TBIL, AST, ALT and creatinine may suggest potential hepatotoxicity and nephrotoxicity at 15 mg/kg. The decrease in platelets and lymphocytes count in the hematology analysis may also suggest potential immunotoxicity at 15 mg/kg. Histopathology analysis of the collected tissues identified some lesions in the liver, kidney, thymus and spleen. However, these lesions were present in a very mild form after treatment and may be considered as adaptive changes.

Collectively, the NOAEL (No Observed Adverse Effect Level) is estimated at 5 mg/kg with potential toxicity occurring at dose level of 15 mg/kg. Based on the presence of lesions and significant changes in clinical chemistry parameters, liver, kidney, spleen and thymus were most likely the primary target organs of toxicity. The MTD (Maximum Tolerated Dose) of cIgG_{B}_12B6FcX-MMAE could not be determined due to lack of significant behaviorial abnormalities, weight loss and mortality in this study.

1. An antigen-binding molecule that specifically binds to Myosin Heavy Chain 9 (MYH9).

2. The antigen-binding molecule of claim 1, wherein the MYH9 is canine MYH9.

3. The antigen-binding molecule of claim 1 or 2, wherein the MYH9 is a glycosylated MYH9.

4. The antigen-binding molecule of any one of claims 1 to 3, wherein the antigen-binding molecule comprises:
a) a heavy chain variable (VH) region comprising the VHCDR1 amino acid sequence GYSITSDYAWN (SEQ ID NO: 1), the VHCDR2 amino acid sequence YISYSGSTNYNPSLKS (SEQ ID NO: 2) and the VHCDR3 amino acid sequence NPPFVY (SEQ ID NO: 3); and
b) a light chain variable (VL) region comprising the VLCDR1 amino acid sequence TASSGVSSGYLH (SEQ ID: 4), the VLCDR2 amino acid sequence STSNLAS (SEQ ID NO: 5) and the VLCDR3 amino acid sequence HQYHRSPFT (SEQ ID NO: 6).

5. The antigen-binding molecule of any one of claims 1 to 4, wherein the antigen-binding molecule comprises:
a) a VH region comprising an amino acid sequence having at least 70% sequence identity to QVQLQESGPGLVKPSQSLSLTCTVTGYSITSDYAWNWLRQFPGNKLEWM GYISYSGSTNYNPSLKSRISITRDTSKNQFFLQLNSVTTEDTATYYCARNPP FVYWGQGTLVTVST (SEQ ID NO: 7); and
b) a VL region comprising an amino acid sequence having at least 70% sequence identity to
(SEQ ID NO: 8)
QIVLTQSPAIMSASLGERVTMTCTASSGVSSGYLHWYQQKPGSSPKLWIY
STSNLASGVPARFSGSGSGTSYSLTISSMEAEDAATYYCHQYHRSPFTFG
SGTKLEIERADAAPTVS.

6. The antigen-binding molecule of any one of claims 1 to 5, wherein the antigen binding molecule is an antibody or antigen-binding fragment thereof.

7. The antigen-binding molecule of claim 6, wherein the antibody or antigen-binding molecule thereof is caninized or chimerized.

8. The antigen-binding molecule of claim 6 or 7, wherein the antibody or antigen binding fragment thereof is a full-length antibody, a substantially intact antibody, a Fab fragment, a scFab, a Fab′, a single chain variable fragment (scFv) or a one-armed antibody.

9. The antigen-binding molecule of any one of claims 1-8, wherein the antigen-binding molecule is capable of being internalized into a cell.

10. A chimeric molecule comprising an antigen-binding molecule according to any one of the preceding claims and a heterologous moiety.

11. The chimeric molecule of claim 10, wherein the heterologous moiety is a detectable moiety, a half-life extending moiety or a therapeutic moiety.

12. The chimeric molecule of claim 11, wherein the therapeutic moiety is a toxin.

13. The chimeric molecule of claim 12, wherein the toxin is auristatin or saporin.

14. The chimeric molecule of any one of claims 10-13, wherein the chimeric molecule is capable of being internalized into a cell.

15. An isolated polynucleotide comprising a nucleic acid sequence encoding the antigen-binding molecule according to any one of claims 1 to 9, or the chimeric molecule of any one of claims 10 to 14.

16. A construct comprising a polynucleotide of claim 15 in operable connection with one or more control sequences.

17. A host cell that contains the construct of claim 16.

18. A pharmaceutical composition comprising an antigen-binding molecule according to any one of claims 1 to 9 or a chimeric molecule according to any one of claims 10 to 14.

19. An antigen-binding molecule according to any one of claims 1 to 9, a chimeric molecule according to any one of claims 10 to 14 or a pharmaceutical composition according to claim 18 for use as a medicament.

20. A method for reducing or inhibiting proliferation and/or viability of a cancer cell, the method comprising contacting the cancer cell with a therapeutically effective amount of an antigen-binding molecule according to any one of claims 1 to 9, a chimeric molecule according to any one of claims 10 to 14 or a pharmaceutical composition according to claim 18, wherein the cancer cell is a cancer cell selected from a mast cell tumor, a mammary carcinoma, a hepatocellular carcinoma, a urothelial carcinoma, a histiocytic sarcoma, a Leydig cell tumor or a seminoma.

21. A method of reducing or inhibiting proliferation, survival and/or viability of a cancer in a subject, the method comprising administering a therapeutically effective amount of an antigen-binding molecule according to any one of claims 1 to 9, a chimeric molecule according to any one of claims 10 to 14 or a pharmaceutical composition according to claim 18 to the subject, wherein the cancer is selected from a mast cell tumor, a mammary carcinoma, a hepatocellular carcinoma, a urothelial carcinoma, a histiocytic sarcoma, a Leydig cell tumor or a seminoma.

22. The method of claim 21, wherein the subject is a canine subject.

23. A method of treating a cancer in a subject, the method comprising administering a therapeutically effective amount of an antigen-binding molecule according to any one of claims 1 to 9, a chimeric molecule according to any one of claims 10 to 14 or a pharmaceutical composition according to claim 18 to the subject, wherein the cancer is selected from a mast cell tumor, a mammary carcinoma, a hepatocellular carcinoma, a urothelial carcinoma, a histiocytic sarcoma, a Leydig cell tumor or a seminoma.

24. A method of treating a disease or condition associated with an undesired expression of MYH9 in a subject, wherein the method comprises administering a therapeutically effective amount of an antigen-binding molecule according to any one of claims 1 to 9, a chimeric molecule according to any one of claims 10 to 14 or a pharmaceutical composition according to claim 18 to the subject.

25. A method of detecting the likelihood of the presence of a cancer in a subject, the method comprising determining the level of MYH9 in a sample obtained from the subject, wherein an increased level of MYH9 as compared to a reference indicates the likelihood of the presence of a cancer in the subject, wherein the cancer is selected from a mast cell tumor, a mammary carcinoma, a hepatocellular carcinoma, a urothelial carcinoma, a histiocytic sarcoma, a Leydig cell tumor or a seminoma.

26. The method of claim 25, wherein the sample is a cell, tissue or blood sample.

27. The method of claim 25 or 26, wherein the method comprises contacting the sample with an antigen-binding molecule according to any one of claims 1 to 9 or a chimeric molecule according to any one of claims 10 to 14 to determine the level of MYH9 in the sample.

28. A method of predicting the prognosis of a cancer in a subject, the method comprising determining the level of MYH9 in a sample obtained from the subject, wherein an increased level of MYH9 as compared to a reference indicates a likelihood of a poor prognosis associated with tumor invasiveness in the subject, wherein the cancer is selected from a mast cell tumor, a mammary carcinoma, a hepatocellular carcinoma, a urothelial carcinoma, a histiocytic sarcoma, a Leydig cell tumor or a seminoma.


SEQUENCE LISTING (13 sequences)

SEQ ID NO: 1 - protein, 11 aa, Artificial Sequence
  VHCDR1
GYSITSDYAWN

SEQ ID NO: 2 - protein, 16 aa, Artificial Sequence
  VHCDR2
YISYSGSTNYNPSLKS

SEQ ID NO: 3 - protein, 6 aa, Artificial Sequence
  VHCDR3
NPPFVY

SEQ ID NO: 4 - protein, 12 aa, Artificial Sequence
  VLCDR1
TASSGVSSGYLH

SEQ ID NO: 5 - protein, 7 aa, Artificial Sequence
  VLCDR2
STSNLAS

SEQ ID NO: 6 - protein, 9 aa, Artificial Sequence
  VLCDR3
HQYHRSPFT

SEQ ID NO: 7 - protein, 115 aa, Artificial Sequence
  Heavy chain variable region
QVQLQESGPGLVKPSQSLSLTCTVTGYSITSDYAWNWLRQFPGNKLEWMGYISYSGSTNYNPSLKSRISITRDTSKNQFFLQLNSVTTEDTATYYCARNPPFVYWGQGTLVTVST

SEQ ID NO: 8 - protein, 117 aa, Artificial Sequence
  Light chain variable region
QIVLTQSPAIMSASLGERVTMTCTASSGVSSGYLHWYQQKPGSSPKLWIYSTSNLASGVPARFSGSGSGTSYSLTISSMEAEDAATYYCHQYHRSPFTFGSGTKLEIERADAAPTVS

SEQ ID NO: 9 - protein, 450 aa, Artificial Sequence
  Heavy chain
QVQLQESGPGLVKPSQSLSLTCTVTGYSITSDYAWNWLRQFPGNKLEWMGYISYSGSTNYNPSLKSRISITRDTSKNQFFLQLNSVTTEDTATYYCARNPPFVYWGQGTLVTVSTASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 10 - protein, 218 aa, Artificial Sequence
  Light chain
QIVLTQSPAIMSASLGERVTMTCTASSGVSSGYLHWYQQKPGSSPKLWIYSTSNLASGVPARFSGSGSGTSYSLTISSMEAEDAATYYCHQYHRSPFTFGSGTKLEIERTDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 11 - protein, 1960 aa, Artificial Sequence
  Canis lupus familiaris Myosin-9
  misc_feature (104)..(104): Xaa can be any naturally occurring amino acid
MAQQAADKYLYVDKNFINNPLAQADWAAKKLVWVPSDKSGFEPASLKEEVGEEAIVELVENGKKVKVNKDDIQKMNPPKFSKVEDMAELTCLNEASVLHNLKEXYYSGLIYTYSGLFCVVINPYKNLPIYSEEIVEMYKGKKRHEMPPHIYAITDTAYRSMMQDREDQSILCTGESGAGKTENTKKVIQYLAHVASSHKSKKDQGELERQLLQANPILEAFGNAKTVKNDNSSRFGKFIRINFDVNGYIVGANIETYLLEKSRAIRQAKEERTFHIFYYLLSGAGEHLKTDLLLEPYNKYRFLSNGHVTIPGQQDKDMFQETMEAMRIMGIPEEEQMGLLRVISGVLQLGNIVFKKERNTDQASMPDNTAAQKVSHLLGINVTDFTRGILTPRIKVGRDYVQKAQTKEQADFAIEALAKATYERMFRWLVLRINKALDKTKRQGASFIGILDIAGFEIFDLNSFEQLCINYTNEKLQQLFNHTMFILEQEEYQREGIEWNFIDFGLDLQPCIDLIEKPAGPPGILALLDEECWFPKATDKSFVEKVVQEQGTHPKFQKPKQLKDKADFCIIHYAGKVDYKADEWLMKNMDPLNDNIATLLHQSSDKFVSELWKDVDRIIGLDQVAGMSETALPGAFKTRKGMFRTVGQLYKEQLAKLMATLRNTNPNFVRCIIPNHEKKAGKLDPHLVLDQLRCNGVLEGIRICRQGFPNRVVFQEFRQRYEILTPNSIPKGFMDGKQACVLMIKALELDSNLYRIGQSKVFFRAGVLAHLEEERDLKITDVIIGFQACCRGYLARKAFAKRQQQLTAMKVLQRNCAAYLKLRNWQWWRLFTKVKPLLQVSRQEEEMMAKEEELVKVREKQLAAENRLTEMETLQSQLMAEKLQLQEQLQAETELCAEAEELRARLTAKKQELEEICHDLEARVEEEEERCQHLQAEKKKMQQNIQELEEQLEEEESARQKLQLEKVTTEAKLKKLEEDQIIMEDQNCKLAKEKKLLEDRIAEFTTNLMEEEEKSKSLAKLKNKHEAMITDLEERLRREEKQRQELEKTRRKLEGDSTDLNDQIAELQAQIAELKMQLAKKEEELQAALARVEEEATQKNMALKKIRELESQISELQEDLESERASRNKAEKQKRDLGEELEALKTELEDTLDSTAAQQELRSKREQEVNILKKTLEEEARTHEAQIQEMRQKHSQAVEELAEQLEQTKRVKANLEKAKQTLENERGELANEVKVLQQGKGDSEHKRKKAEAQLQELQVKFTEGERVRTELADKVTKLQVELDNVMGLLTQSDSKSSKLTKDFSALESQLQDTQELLQEENRQKLSLSTKLKQMEDEKNSFKEQLEEEEEAKRNLEKQIATLHAQVTDMKKKMEDGVGCLETAEEAKRKLQKDLEGLGQRYEEKVAAYDKLEKTKTRLQQELDDLLVDLDHQRRTASNLEKKQKKFDQLLAEEKTISAKYAEERDRAEAEAREKETKALSLARALEEAMEQKAELERLNKQFRTEMEDLMSSKDDVGKSVHELEKSKRALEQQVEEMKTQLEELEDELQATEDAKLRLEVNLQAMKAQFERDLQGRDEQSEEKKKQLVRQVREMEAELEDEKKQRSMAVAARKKLEMDLKDLEAHIDSANKNRDEAIKQLRKLQAQMKDCVRELDDTRASREEILAQAKENEKKMKSMEAEMIQLQEELAAAERAKRQAQQERDELADEIANSSGKGALALEEKRRLEARIAQLEEELEEEQGNTELVNDRLKKANLQIDQINTDLNLERSHAQKNENARQQLERQNKELKVKLQEMEGTVKSKYKASITALEAKIAQLEEQLDNETKERQAACKQVRRAEKKLKDVLLQVDDERRNAEQFKDQADKASTRLKQLKRQLEEAEEEAQRANASRRKLQRELEDATETADAMNREVSSLKNKLRRGDLPFVVPRRVARKGAGDCSDEEVDGKADGAEAKAAE

SEQ ID NO: 12 - DNA, 345 bp, Artificial Sequence
  Nucleotide sequence of heavy chain variable region of 12B6
caggtcaagctgcaggagtcaggacctggcctggtgaaaccttctcagtctctgtccctcacctgcactgtcactggctactcaatcaccagtgattatgcctggaactggatccggcagtttccaggaaacaaactggagtggatgggctacataagctacagtggtagcactaactacaacccatctctcaaaagtcgaatctctatcactcgagacacatccaagaaccagttcttcctgcagttgaattctgtgactactgaggacacagccacatattactgtgcaaggaaccccccgtttgtttactggggccaagggaccacggtcaccgtctcctca

SEQ ID NO: 13 - DNA, 350 bp, Artificial Sequence
  Nucleotide sequence of light chain variable region of 12B6
caaattgttctcacccagtctccagcaatcatgtctgcatctctaggggaacgggtcaccatgacctgcactgccagctcaggtgtaagttccggttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccattcacgttcggctcggggacaaagttggaaatagaacgggctgatgctgcaccaactgtatc